(19) United States
(12) Patent Application Publication
(10) Pub. No.: US 2003/0087273 A1
(43) Pub. Date: May 8, 2003

(54) Compositions and methods for inhibiting human immunodeficiency virus infection by down-regulating human cellular genes

(76) Inventors: Tanya A. Holzmayer, Mountain View, CA (US); Andrew Holzmayer, Libertyville, IL (US); Stephen J. Dunn, Mountain View, CA (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/186,593

(22) Filed: Jul. 1, 2002

Related U.S. Application Data
(60) Provisional application No. 60/302,157, filed on Jun. 29, 2001.
(60) Provisional application No. 60/313,252, filed on Aug. 17, 2001.

(51) Int. Cl. A61K 31/00; C12Q 1/70; C12Q 1/68; A01N 61/00

(52) U.S. Cl. 435/6; 435/5; 514/1

(57) ABSTRACT

The invention provides methods for identifying human cellular genes and their encoded products for use as targets in the design of therapeutic agents for inhibiting or suppressing human immunodeficiency virus (HIV) infection. The invention also provides methods for identifying protective compounds including immunizing agents that inhibit HIV infection. The invention further provides compounds for use in the treatment or prevention of HIV.

This application claims priority to U.S. Provisional Patent Applications, Serial No. 60/ 302,157, filed Jun. 29, 2001 and Serial No. 60/313,252, filed Aug. 17, 2001, the entire disclosure of each of which is explicitly incorporated by reference herein.

### BACKGROUND OF THE INVENTION

1. Field of the Invention

The invention relates to methods for identifying human cellular genes and their encoded products for use as targets in the design of therapeutic agents for suppressing human immunodeficiency virus (HIV) infection. In particular, the invention relates to methods for identifying biochemical pathways, substrates and metabolic products of said pathways, and enzymes that mediate conversion of substrates into metabolic products, wherein said pathways comprise one or a plurality of targets for the design of preventative and therapeutic agents for preventing, inhibiting, suppressing or immunizing against infection of naive cells with HIV or production of infectious virus from infected cells. The invention also relates to methods for identifying protective compounds that inhibit HIV infection. The invention further relates to compounds for use in the treatment or prevention of HIV.

2. Background of the Invention

The primary cause of acquired immunodeficiency syndrome (AIDS) has been shown to be HIV (Barre-Sinoussi et al., 1983, Science 220:868-70; Gallo et al., 1984, Science 224:500-03). HIV causes immunodeficiency in an individual by infecting important cell types of the immune system, which results in the depletion of these cells. This, in turn, leads to opportunistic infections, neoplastic growth, and death.

HIV is a member of the lentivirus family of retroviruses (Teich et al., 1984, in RNA Tumor Viruses pp. 949-56 (Weiss et al., eds., CSH-Press: New York). Retroviruses are small, enveloped viruses that contain a diploid, single-stranded RNA genome, and replicate via a DNA intermediate produced by a virally encoded reverse transcriptase, an RNA-dependent DNA polymerase (Varmus, 1988, Science 240:1427-39). There are at least two distinct subtypes of HIV: HIV-1 (Barre-Sinoussi et al., 1983, ibid.; Gallo et al., 1984, ibid.) and HIV-2 (Clavel et al., 1986, Science 233:343-46; Guyader et al., 1987, Nature 326:662-69). Genetic heterogeneity exists within each of these HIV subtypes.

CD4⁺ cells, such as T cells, macrophages and dendritic cells, are targets for HIV-1 infection because the CD4 cell surface protein acts as the main cellular receptor for HIV attachment (Kalter et al., 1991, Dermatol. Clin. 9:415-28). CD4⁺ T cells represent the predominant targets of HIV and infection of these cells is associated with progression to disease (Dalgleish et al., 1984, Nature 312:763-67; Klatzmann et al., 1984, Nature 312:767-68; Maddon et al., 1986, Cell 47:333-48; Connor et al., 1993, J. Virol. 67:1772-77)

HIV infection is pandemic and HIV-associated diseases have become a worldwide health problem. Despite considerable efforts in the design of anti-HIV modalities, there is, thus far, no successful prophylactic or therapeutic regimen against AIDS. However, several stages of the HIV life cycle have been considered as potential targets for therapeutic intervention (Mitsuya et al., 1991, FASEB J. 5:2369-81).

For example, virally encoded reverse transcriptase has been a major focus of drug development. A number of reverse transcriptase-targeted drugs, including dideoxynucleotide analogs such as AZT, ddI, ddC, and ddT have been shown to be active against HIV (Mitsuya et al., 1990, Science 249:1533-44). While beneficial, these nucleotide analogs are not curative, probably due to the rapid appearance of drug resistant HIV mutants (Lander et al., 1989, Science 243:1731-34). In addition, these drugs often exhibit toxic side effects, such as bone marrow suppression, vomiting, and liver abnormalities.

Another stage of the HIV life cycle that has been targeted is viral entry into cells, the earliest stage of HIV infection. Viral entry into cells is dependent upon the binding of viral protein gp120 to the cellular CD4 receptor molecule as well as one of several chemokine receptors, such as CCR2, CCR3, CCR5 or CXCR-4, followed by virus-cell membrane fusion (McDougal et al., 1986, Science 231:382-85; Maddon et al., 1986, Cell 47:333-48; Moore, 1997, Science 276:51-52; Cohen, 1997, Science 275:1261). The binding of the virus to CD4 and the chemokine receptor as well as virus-cell membrane fusion have been targeted for antivirals. Recombinant soluble CD4 protein has been utilized to inhibit infection of CD4⁺ T cells by some HIV-1 strains (Smith et al., 1987, Science 238:1704-07). Certain primary HIV-1 isolates, however, are relatively less sensitive to inhibition by recombinant CD4 (Daar et al., 1990, Proc. Natl. Acad. Sci. U.S.A. 87:6574-79). Clinical trials of recombinant, soluble CD4 have produced disappointing results (Schooley et al., 1990, Ann. Int. Med. 112:247-53; Kahn et al., 1990, Ann. Int. Med. 112:254-61; Yarchoan et al., 1989, Proc. Vth Int. Conf on AIDS 564, MCP 137; Arthos et al., 2002, J. Biol. Chem. 277:11456-64). Chemokine receptors present an additional cellular target for the design of HIV therapeutic agents. Chemokine receptor inhibitors, both small molecule and peptide derivatives of chemokine ligands, are being tested as anti-HIV agents (D'Souza et al., 2000, JAMA 284:215-222). Inhibition of entry has also been achieved by blocking virus-cell membrane fusion using modalities such as T-20, a synthetic peptide derived from heptad repeats of gp41 (Kilby et al., 1998, Nat. Med. 4:1302-1307).

Yet another stage of the HIV life cycle that has been targeted is the integration of the proviral DNA into the host genome. The viral enzyme integrase catalyzes the process of integration, and inhibitors of integrase have been reported (d'Angelo et al., 2001, Pathol. Biol. 49:237-46; Farnet and Bushman, 1996, AIDS 10 Supp. A:S3-11).

Additionally, the later stages of HIV replication (which involve crucial virus-specific processing of certain viral proteins and enzymes) have been targeted for anti-HIV drug development. Late-stage processing is dependent on the activity of a virally encoded protease, and drugs including saquinavir, ritonavir, and indinavir have been developed to inhibit this protease (Pettit et al., 1993, Persp. Drug Discov. Design 1:69-83). With this class of drugs, the emergence of drug resistant HIV mutants is also a problem; resistance to one inhibitor often confers cross-resistance to other protease inhibitors (Condra et al., 1995, Nature 374:569-71). Also, these drugs often exhibit toxic side effects such as nausea, altered sense of taste, circumoral parethesias, development of lipodystrophy, diarrhea, and nephrolithiasis.

Antiviral therapy of HIV using different combinations of nucleoside analogs and protease inhibitors (highly active anti-retroviral treatment, HAART) have been shown to be more effective than the use of a single drug alone (Torres et al., 1997, Infec. Med. 14:142-60). However, despite the ability to achieve significant decreases in viral burden, there is no evidence to date that combinations of available drugs will afford a curative treatment for AIDS.

Other potential approaches for developing treatment for AIDS include the delivery of exogenous genes into infected cells. One such gene therapy approach involves the use of genetically engineered viral vectors to introduce toxic gene products to kill HIV-infected cells. Another form of gene therapy is designed to protect virally infected cells from cytolysis by specifically disrupting viral replication. Stable expression of RNA-based HIV-1 antiviral agents (e.g., decoys, antisense, or ribozymes) or protein-based HIV-1 antiviral agents (e.g., transdominant mutants) can inhibit certain stages of the viral life cycle. A number of anti-HIV suppressors have been reported, such as decoy RNA of TAR or RRE (Sullenger et al., 1990, Cell 63:601-08; Sullenger et al., 1991, J. Virol. 65:6811-16; Lisziewicz et al., 1993, New Biol. 3:82-89; Lee et al., 1994, J. Virol. 68:8254-64), antisense RNA complementary to the mRNA of viral gag, tat, rev, or env genes (Sezakiel et al., 1991, J. Virol. 65:468-72; Chatterjee et al., 1992, Science 258:1485-88; Rhodes et al., 1990, J. Gen. Virol. 71:1965; Rhodes et al., 1991, AIDS 5:145-51; Sezakiel et al., 1992, J. Virol. 66:5576-81; Joshi et al., 1991, J. Virol. 65:5524-30) and transdominant mutants of Rev (Bevec et al., 1992, Proc. Natl. Acad. Sci. U.S.A. 89:9870-74), Tat (Pearson et al., 1990, Proc. Natl. Acad. Sci. U.S.A. 87:5079-83; Modesti et al., 1991, New Biol. 3:759-68), Gag (Trono et al., 1989, Cell 59:113-20), Env (Bushschacher et al., 1995, J. Virol. 69:1344-48) and protease (Junker et al., 1996, J. Virol. 70:7765-72; Todd et al., 2000, Biochim Biophys Acta 1477:168-88).

Antisense polynucleotides have been designed to complex with and sequester HIV-1 transcripts (International Pub. Nos. WO 93/11230 and WO 94/10302; European Patent Pub. No. EP 594,881; Chatterjee et al., 1992, Science 258:1485). Furthermore, enzymatically active RNAs (i.e., ribozymes) have been used to cleave viral transcripts. The use of a ribozyme to generate resistance to HIV-1 in a hematopoietic cell line has been reported (Ojwang et al., 1992, Proc. Natl. Acad. Sci. U.S.A. 89:10802-06; Yamada et al., 1994, Gene Therapy 1:38-45; International Pub. Nos. WO 94/26877 and WO 95/13379). In preclinical studies, RevM10, a transdominant Rev protein, has been transfected ex vivo into CD4⁺ cells of HIV-infected individuals and shown to confer survival advantage over cells transfected with vector alone (Woffendin et al., 1996, Proc. Natl. Acad. Sci. U.S.A. 93:2889-94).

However, despite enormous efforts in the art, reliable, curative anti-HIV therapeutic agents and regimens have not been developed.

In nature, evolution of an intracellular pathogen such as HIV requires the development of interactions of its genes and-gene products with multiple cellular components. For instance, the interactions of a virus with a host cell involves the binding of the virus to specific cellular receptors, translocation through the cellular membrane, uncoating, replication of the viral genome, and transcription of the viral genes. Each of these events occurs in a cell and involves interactions with at least one cellular component. Thus, the life cycle of a virus can be completed only if the cell is “permissive” for viral infection. The availability of amino acids and nucleotides for replication of the viral genome and protein synthesis, the energy status of the cell, and the presence of cellular transcription factors and enzymes all contribute to the propagation of the virus in the cell. Consequently, the cellular components, in part, determine host cell susceptibility to infection and can be used as potential targets for the development of new therapeutic interventions. In the case of HIV, one cellular component that has been used towards this end is the cell surface molecule for HIV attachment, CD4.

Thus, there remains a need for the discovery of additional cellular targets for the design of anti-HIV therapeutics, particularly intracellular targets for disrupting viral replication after viral entry into a cell. There remains a need in the art to isolate and identify human cellular genes that encode products that are necessary for productive HIV infection for use as targets in the design of therapeutic agents for suppressing HIV infection. There also remains a need in the art to identify the biological pathways comprising the products of such cellular genes. There further remains a need in the art to isolate and identify additional human cellular genes that encode products comprising other members of such biological pathways for use as targets in the design of therapeutic agents for suppressing HIV infection. The identification of human cellular genes that encode products that are necessary for productive HIV infection, biological pathways comprising the products of such cellular genes, and additional human cellular genes that encode products comprising other members of such biological pathways, would allow for the identification of novel protective compounds that inhibit, suppress or otherwise interfere with HIV infection.

### SUMMARY OF THE INVENTION

The invention relates to methods for identifying human cellular genes that encode products that are necessary for productive HIV infection for use as targets in the design of therapeutic agents for suppressing HIV infection. The invention also relates to methods for identifying biological pathways comprising the products of such cellular genes, as well as substrates and metabolic products of said pathways. The invention further relates to methods for identifying additional human cellular genes that encode products comprising other members of such biological pathways for use as targets in the design of therapeutic agents for suppressing HIV infection.

The invention also relates to methods for identifying protective compounds that inhibit HIV infection. The invention further relates to compounds for use in the treatment or prevention of HIV.

In one embodiment of the methods of the invention is provided methods for identifying a compound capable of inhibiting HIV infection in a cell comprising the step of identifying an inhibitor of a target in said human host cell, wherein said target is URF6, URF 2, Squalene synthetase, RTLV associated endogenous retrovirus, Human 2-oxoglutarate dehydrogenase, TCBA, Calnexin, HAUSP, ARF3, eIF4B, eIF3, Glucosidase II, Glucosidase II, Na⁺-D-glucose cotransport regulator, CD47, CD44, BDP-1 tyrosine phosphatase, P13K, EF-1, Mitochondrial aspartate amino transferase, Double strand break repair gene, guanine nucleotide releasing protein, BTG-1, Lymphocyte specific protein 1, Protein phosphatase 2A, ERF-1, GTP binding protein, Importin beta subunit, L1CAM, HSPG, Zinc finger factor 1, BMP1-6, U-snRNP associated cyclophilin, Recepin, Lipocortin II/Annexin II, hnRNP A1, ArgBP2a, Keratin related protein, Glucosyltransferase, Rox, p18 protein, Elc, Ferritin heavy subunit, p40, MIP-1α, HSP90, MIP-1β, NF-kB binding subunit, BBC-1, α-enolase, TCTP, DAP 5, FK-506 binding protein 1A, TRAP-beta, TID1, HIP, PABP, Cytokine effector-inflammatory response, Nuclear U4A RNA, HnRNP A2/B1, IL-1 beta, TNF-α receptor, HYPK mRNA, HIV-1 TAR binding protein, TRAP-delta, ATP6E, MO25, CD69, Mitochondrial cytochrome oxidase I, Csa-19, 14-3-3 zeta protein, Nip 7-1, EF-1 delta, E16 mRNA, Arginyl TRNA synthetase, Novel nuclear targeted gene, eIF4AII, WBSCRI, C21orf4, Protein phosphatase 2A B56 gamma 1, DAP12, PDCD4, Glutaredoxin, eIF4AI, GA17, MAD-3/NFKBIA, RANTES, IL-6, FYN binding protein, ABC transporter, HSHIP, IEX-IL, CDC42, Tryptophanyl tRNA synthetase, TRAP-gamma, CXCR-4, Cyclin T1, PDIR, G3PDH, CCR4, GNB2L1, Cathepsin B, Cathepsin L, Vacuolar H+ ATPase proton channel subunit 6C, Prolyl 4-hydroxylase, Protein phosphatase 2A α catalytic, ATP1A1, O-linked GlcNAc transferase, CDP-diacylglycerol synthase 2, FoF1 ATP synthase f subunit, Guanylate binding protein, ATP5G2, Phosphorylase kinase, alpha 2, SOD-2, NADH ubiquinone oxidoreductase B22 subunit, DEAD/H Box 5, DEAD/H 9, Aryl Sulfotransferase, Cytochrome b gene, ATIC, Cytochrome bc-1 core protein, CD 11c, HELO1, NPM-RAR, Protein phosphatase I regulatory, Aldehyde dehydrogenase, Glucosamine-6-, phosphate deaminase, DDX3, ATP5E, CAPNS1, CARM1, CSNK1E, CTSD, CCR7, CD68, CD74, CLK3, CSAD, CSF3R, CSNK1G2, DDXL, DNMT3A, DUSP1, GPRK6, Human ADP/ATP translocase, LENG8, MAP2K7, MIF, MINK, NME4, Nonreceptor protein-tyrosine kinase (fgr), P101-PI3K, P2X1 receptor gene, PDE3B, PTK2B, PTPN23, RAB7, SLC11A1, SMG1, STK10, TAP1, TBXA2R, TYK2, UBE2M, UP, or GABBR1.

In particular, such target can be used to identify the inhibitor using a method comprising: (a) contacting a human host cell with a putative inhibitor; and (b) assessing inhibition of a target by a method including: (i) assaying for reduced expression of the target; and (ii) assaying for reduced activity of the target. It is an advantage of this invention that the range of targets for inhibiting HIV infection is larger than HIV-specific genes and gene products. It is a particular advantage of the invention that a multiplicity of cellular genes and gene products are identified, the inhibition or interference with the function of which inhibits HIV infection. Thus, cellular genes involved in HIV infection are recognized herein as targets for drugs that inhibit or interfere with cellular gene expression of gene product function, thereby provide novel classes of anti-HIV drugs. Specific preferred embodiments of the invention will become evident from the following more detailed description of certain preferred embodiments and the claims.

### DETAILED DESCRIPTION OF THE PREFERRED EMBODIMENTS

The invention includes methods for identifying human cellular genes that encode products that are necessary for productive HIV infection for use as targets in the design of therapeutic agents for suppressing HIV infection. The invention also includes methods for identifying biological pathways comprising the products of such cellular genes. The invention further includes methods for identifying additional human cellular genes that encode products comprising other members of such biological pathways for use as targets in the design of therapeutic agents for suppressing HIV infection.

The invention also includes methods for identifying protective compounds that inhibit HIV infection. The invention further includes compounds for use in the treatment or prevention of HIV. Such compounds include chemical compounds and biological compounds. Chemical compounds or biological compounds include any chemical or biological compound that disrupts or inhibits one or more biological functions required for mediation or replication of HIV. Preferred chemical compounds include small molecule inhibitor or substrate compounds, such as products of chemical combinatorial libraries. Preferred biological compounds include peptides, anti-sense molecules and antibodies.

As used herein, the term “HIV infection” refers to the ability of HIV to enter a host cell and/or replicate in the host cell.

As used herein, the term “isolated nucleic acid molecule” refers to a nucleic acid molecule that has been removed from its natural milieu (i.e., a molecule that has been subject to human manipulation) and can include DNA, RNA, or derivatives of either DNA or RNA. An isolated nucleic acid molecule can be isolated from its natural source or can be produced using recombinant DNA technology (e.g., polymerase chain reaction amplification) or chemical synthesis. Isolated nucleic acid molecules include natural nucleic acid molecules and homologs thereof, including, but not limited to, natural allelic variants and modified nucleic acid molecules in which nucleotides have been inserted, deleted, substituted, or inverted in such a manner that such modifications do not substantially interfere with the nucleic acid molecule's ability to inhibit HIV infection.

It should also be appreciated that reference to an isolated nucleic acid molecule does not necessarily reflect the extent of purity of the nucleic acid molecule. Nucleic acid molecules can be isolated and obtained in substantial purity, generally as other than an intact chromosome. Usually, the nucleic acid molecule will be obtained substantially free of other nucleic acid sequences, generally being at least about 50%, and usually at least about 90% pure. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably.

According to the invention, reference to an “isolated nucleic acid molecule” refers to a nucleic acid molecule that is the size of or smaller than a gene. Thus, an isolated nucleic acid molecule does not encompass isolated genomic DNA or an isolated chromosome. The term isolated nucleic acid molecule does not connote any specific minimum length. As used herein, the term “gene” has the meaning that is well known in the art, that is, a nucleic acid sequence that includes the translated sequences that code for a protein (“exons”) and the untranslated intervening sequences (“introns”), and any regulatory elements ordinarily necessary to translate the protein.

“Hybridization” has the meaning that is well known in the art, that is, the formation of a duplex structure by two single-stranded nucleic acids due to complementary base pairing. Hybridization can occur between exactly complementary nucleic acid strands or between nucleic acid strands that contain some regions of mismatch. “Stringent hybridization” has a meaning well-established in the art, that is, hybridization performed at a salt concentration of no more than 1M and a temperature of at least 25 degrees Celsius. For example, conditions of 5×SSPE (750 mM NaCl, 50 mM Sodium Phosphate, 5 mM EDTA, pH 7.4) and a temperature of 55 degrees to 60 degrees Celsius are suitable. “Moderately stringent conditions” can be defined as hybridizations carried out as described above, followed by washing in 0.2×SSC and 0.1% SDS at 42 degrees Celsius (Ausubel et al., 1989, Current Protocols for Molecular Biology, ibid.).

As used herein, the term “validated target” means Target that has been shown to be involved in the occurrence of a biological phenotype. Methods to validate a Target include affecting the expression of a Target gene, inhibiting the translation of the RNA encoded by such gene or inhibiting the activity of the protein encoded by such RNA. Validation can also include inducing or increasing the expression of a Target gene or RNA, or increasing the activity of a Target protein. In preferred embodiments, the target is validated by a process comprising the steps of:

(a) inhibiting said target in a cell by a method selected from the group consisting of gene knock-out, anti-sense oligonucleotide expression, target overexpression, viral stage assays, GSE expression and Target protein inhibition assays, and

(b) assaying said cell for the ability of HIV to infect said cell.

Viral stage assays refer to assays that determine at which stage in the HIV lifecycle a particular Target is expressed or involved. Target protein inhibition assays include using any reagent suitable for blocking the activity of a Target protein, such reagents including but not limited to chemical compounds, antibodies, peptides, substrate analogs and any other reagent that binds to a protein in such a manner to inhibit the protein's intended binding, enzymatic or other activity.

The invention is based, in part, on the Applicants' discovery that certain nucleic acid molecules—termed genetic suppressor elements (GSEs)—can be isolated from human cells that prevent activation of latent HIV-1 in a CD4⁺ cell line as well as productive HIV infection in such cells, and that such nucleic acid molecules correspond to fragments of certain human cellular genes. In that regard, any cellular or viral marker associated with HIV infection can be used to select for such nucleic acid molecules. An example of such a marker is CD4, which is conveniently monitored by using a specific antibody. Additional markers include virus-specific gene products, such as gp120 and p24.

GSEs having the ability to inhibit HIV infection can be isolated that are functional in the sense orientation (and encode a peptide thereby), and also GSEs that are functional in the antisense orientation (and encode antisense RNAs thereby). These GSEs are believed to down-regulate the corresponding cellular gene from which they were derived by different mechanisms. Such a corresponding cellular gene is referred to herein as a “Target gene” and its product is referred to as a “Target product.” Sense-oriented GSEs exert their effects as transdominant mutants or RNA decoys. Transdominant mutants are expressed proteins or peptides that competitively inhibit the normal function of a wild-type protein in a dominant fashion. RNA decoys are protein binding sites that titrate out these wild-type protein. Anti-sense oriented GSEs exert their effects as antisense RNA molecules, i.e., nucleic acid molecules complementary to the mRNA of the target gene. These nucleic acid molecules bind to mRNA and block the translation of the mRNA. In addition, some antisense nucleic acid molecules can act directly at the DNA level to inhibit transcription.

In one embodiment of the invention, down-regulation of the concentration or activity of a Target gene or product by a GSE depletes a cellular component required for progression through the HIV life cycle resulting in an inhibition of HIV infection. In another embodiment of the invention, down-regulation of the concentration or activity of one Target gene or product by a GSE depletes a cellular component that interacts with another human cellular gene or gene product that encodes a polypeptide required for progression through the HIV life cycle resulting in an inhibition of HIV infection. In a preferred embodiment of the invention, the two human cellular genes are members of the same biological pathway and one human cellular gene or gene product regulates the expression or activity of the other human cellular gene or gene product. In another preferred embodiment of the invention, the two human cellular genes are members of the same biological pathway and the substrate of a biochemical reaction catalyzed by a polypeptide encoded by one human cellular gene is a product of a biochemical reaction mediated by the polypeptide encoded by the other human cellular gene. In still another preferred embodiment of the invention, the two human cellular genes are members of the same biological pathway and the product of a biochemical reaction mediated by the polypeptide encoded by one human cellular gene is a substrate of a biochemical reaction mediated by the polypeptide encoded by the other human cellular gene. In another embodiment, the two human cellular genes encode polypeptides that are isozymes of each other. In a preferred embodiment, at least one of the human cellular genes encodes an enzyme.

It will be understood that this invention is not limited to the particular methodology, protocols, cell lines, animal species or genera, constructs, or reagents described herein, as such may vary. It is also to be understood that the terminology used herein is for the purpose of describing particular embodiments only, and is not intended to limit the scope of the invention that will be limited only by the appended claims. All technical and scientific terms used herein have the same meaning as commonly understood to one of ordinary skill in the art to which this invention belongs unless clearly indicated otherwise.

Target genes or proteins identified using GSEs can be further evaluated using a variety of methods. Such methods include in vivo genetic methods, such as the production of homozygous null or deletion mutant animals, preferably rodents and most preferably mice, and methods that disrupt or “knock out” the expression of a Target gene in a cell capable of being infected with HIV. Knock-out methods include somatic cell knock-outs and inhibitory RNA molecules including anti-sense oligonucleotides, siRNA molecules and RNA decoys, or methods that include nucleic acid-based experiments such as Northern Blots, Real Time polymerase chain reaction or high density microarrays.

Once one or more members of a biological pathway are identified, as required for the progression through the HIV life cycle, it is within the skill of one in the art to identify additional members of a biological pathway that are also required for the progression through the HIV life cycle. For example, the activity of a member of a pathway can be inhibited using methods known to those in the art such as known chemical inhibitors, antibodies, somatic cell gene knock-outs, anti-sense molecules or ribozymes in a cell capable of being infected with HIV. The cell can then be exposed to HIV and HIV infection measured. Inhibition of HIV infection identifies such pathway member as being required for the progression through the HIV life cycle. Methods for testing HIV infection are described in the Examples herein.

The present invention includes a method of identifying an inhibitor of a member of a biological pathway in a human host cell, wherein the member of the biological pathway is necessary for productive HIV infection, the method comprising the steps of: (a) identifying the member of the biological pathway by: (i) synthesizing a randomly fragmented cDNA population from total mRNA isolated from a human host cell that is susceptible to HIV infection to yield DNA fragments; (ii) transferring the DNA fragments to an expression vector to yield a genetic suppressor element library, wherein each of the DNA fragments is operatively linked to a protein translation initiation codon, and wherein the expression vector expresses the DNA fragments in the human host cell; (iii) genetically modifying a first population of human host cells by introducing the genetic suppressor element library into the first population of human host cells; (iv) infecting the first population of human host cells with HIV; (v) isolating a genetically modified human host cell containing a genetic suppressor element conferring resistance to HIV infection from the first population of human host cells; (vi) recovering the genetic suppressor element from the isolated genetically modified human host cell; (vii) determining the human cellular gene corresponding to the genetic suppressor element; and (viii) determining the polypeptide encoded by the human cellular gene; and (b) identifying the inhibitor of the member of the biological pathway by: (i) exposing a second population of human host cells to a test compound; (ii) measuring expression of the polypeptide of step (a)(viii) in the second population of human host cells; and (iii) determining whether the test compound decreases the expression of the member of the biological pathway. In preferred embodiments these methods of the invention are useful for identifying test compounds that inhibit HIV infection in a human cell, comprising the additional steps of (c) contacting a third human host cell population with the compound identified in step (b)(iii), (d) infecting the cells with HIV and (e) identifying compounds that inhibit HIV infection in said third human cell population.

The method can further comprise the step of determining whether the test compound inhibits HIV infection. The expression of the human cellular gene encoding the member of the biological pathway is measured by polymerase chain reaction or using an antibody that specifically recognizes the member of the biological pathway. The activity of the member of the biological pathway is measured by measuring the amount of a product generated in a biochemical reaction mediated by the member of the biological pathway, in particular by measuring the amount of a substrate consumed in a biochemical reaction mediated by the member of the biological pathway.

Once a human cellular gene has been identified as a potential target for supporting the HIV life cycle, an assay can be used for screening and selecting a chemical compound or a biological compound activity as an anti-HIV therapeutic based on the ability to down-regulate expression of the gene or inhibit activity of its gene product. Such compound is referred to herein as therapeutic compound. For example, a cell line that naturally expresses the gene of interest or has been transfected with the gene is incubated with various compounds. A reduction of the expression of the gene of interest or an inhibition of the activities of its encoded product may be used as to identify a therapeutic compound. Therapeutic compounds identified in this manner are then re-tested in other assays to confirm their activities against HIV infection.

Reagents suitable for an assay of the invention include any human cellular gene or its gene product demonstrated to inhibit HIV infection when expression of the gene or activity of the gene product is reduced. Compounds to be screened include those listed herein. Compounds may also be identified using rational drug design relying on the structure of the gene product of a human cellular gene. Such methods are known to those of skill in the art and involve the use of three-dimensional imaging software programs. Compounds can include therapeutic antibodies as well as other biological compounds such as antisense oligonucleotides or peptides.

The invention provides antisense and peptide GSEs that are inhibitors of HIV infection in mammalian, most preferably human cells. In one embodiment of the invention, inhibitors of HIV infection are identified by exposing a mammalian cell to a test compound; measuring the expression of a human cellular gene or an activity of the polypeptide encoded by the human cellular gene in the mammalian cell; and selecting a compound that down-regulates the expression of the human cellular gene or interferes with the activities of its encoded product. A preferred mammalian cell to use in an assay is a mammalian cell that either naturally expresses the human cellular gene or has been transformed with a recombinant form of the human cellular gene. Methods to determine expression levels of a gene are well known in the art.

In a preferred embodiment, the expression of the human cellular gene is measured by the polymerase chain reaction. In another preferred embodiment, the expression of the human cellular gene is measured using an antibody that specifically recognizes the polypeptide encoded by the human cellular gene and is analyzed using methods such as immunoprecipitation, ELISAs, fluorescence activated cell sorting (FACS) and immunofluorescence microscopy. In another embodiment, the expression of the human cellular gene is measured using polyacrylamide gel analysis, chromatography or spectroscopy. In still another preferred embodiment, the activity of the polypeptide encoded by the human cellular gene is measured by measuring the decrease in the amount of substrate, or the increase in the amount of product generated in a biochemical reaction mediated by the polypeptide encoded by the human cellular gene. In still another preferred embodiment, the activity of the polypeptide encoded by the human cellular gene is measured by measuring the amount of substrate generated in a biochemical reaction mediated by the polypeptide encoded by the Target gene. In another embodiment of the invention, therapeutic compounds are selected by determining the three-dimensional structure of a human cellular gene product; and determining the three-dimensional structure of a therapeutic compound. Preferably, the structure of the therapeutic compound is determined using computer software capable of modeling the interaction of a therapeutic compound with the Target gene. One of skill in the art can select the appropriate three-dimensional structure, therapeutic compound, and analytical software based on the identity of the Target gene.

Also provided are related compounds within the understanding of those with skill in the art, such as chemical mimetics, organomimetics or peptidomimetics. As used herein, the terms “mimetic,” “peptide mimetic,” “peptidomimetic,” “organomimetic” and “chemical mimetic” are intended to encompass chemical compounds having an arrangement of atoms is a three-dimensional orientation that is equivalent to that of a compound identified according to the invention. It will be understood that the phrase “equivalent to” as used herein is intended to encompass compounds having substitution of certain atoms or chemical moieties in said compound with moieties having bond lengths, bond angles and arrangements thereof in the mimetic compound that produce the same or sufficiently similar arrangement or orientation of said atoms and moieties to have the biological function of the compounds identified by the methods of the invention and have the HIV infection inhibiting activity thereof. In the mimetics of the invention, the three-dimensional arrangement of the chemical constituents is structurally and/or functionally equivalent to the three-dimensional arrangement of the compounds identified according to the methods of the invention and result in such peptido-, organo- and chemical mimetics having substantial biological activity. These terms are used according to the understanding in the art, as illustrated for example by Fauchere, 1986, Adv. Drug Res. 15: 29; Veber & Freidinger, 1985, TINS p.392; and Evans et al., 1987, J. Med. Chem. 30: 1229, incorporated herein by reference.

It is understood that a pharmacophore exists for the biological activity of each compound identified according to the methods of the invention. A pharmacophore is understood in the art as comprising an idealized, three-dimensional definition of the structural requirements for biological activity. Peptido-, organo- and chemical mimetics can be designed to fit each pharmacophore with current computer modeling software (computer aided drug design). Said mimetics are produced by structure-function analysis, based on the positional information from the substituent atoms in the peptides of the invention.

Sense-oriented GSE peptides as provided by the invention can be advantageously synthesized by any of the chemical synthesis techniques known in the art, particularly solid-phase synthesis techniques, for example, using commercially-available automated peptide synthesizers. The mimetics of the present invention can be synthesized by solid phase or solution phase methods conventionally used for the synthesis of peptides (see, for example, Merrifield, 1963, J. Amer. Chem. Soc. 85: 2149-54; Carpino, 1973, Acc. Chem. Res. 6: 191-98; Birr, 1978, ASPECTS OF THE MERRIFIELD PEPTIDE SYNTHESIS, Springer-Verlag: Heidelberg; THE PEPTIDES: ANALYSIS, SYNTHESIS, BIOLOGY, Vols. 1, 2, 3, 5, (Gross & Meinhofer, eds.), Academic Press: New York, 1979; Stewart et al., 1984, SOLID PHASE PEPTIDE SYNTHESIS, 2nd. ed., Pierce Chem. Co.: Rockford, Ill.; Kent, 1988, Ann. Rev. Biochem. 57: 957-89; and Gregg et al., 1990, Int. J. Peptide Protein Res. 55: 161-214 , which are incorporated herein by reference in their entirety.)

The use of solid phase methodology is preferred. Briefly, an N-protected C-terminal amino acid residue is linked to an insoluble support such as divinylbenzene cross-linked polystyrene, polyacrylamide resin, Kieselguhr/polyamide (pepsyn K), controlled pore glass, cellulose, polypropylene membranes, acrylic acid-coated polyethylene rods or the like. Cycles of deprotection, neutralization and coupling of successive protected amino acid derivatives are used to link the amino acids from the C-terminus according to the amino acid sequence. For some synthetic peptides, an FMOC strategy using an acid-sensitive resin may be used. Preferred solid supports in this regard are divinylbenzene cross-linked polystyrene resins, which are commercially available in a variety of functionalized forms, including chloromethyl resin, hydroxymethyl resin, paraacetamidomethyl resin, benzhydrylamine (BHA) resin, 4-methylbenzhydrylamine (MBHA) resin, oxime resins, 4-alkoxybenzyl alcohol resin (Wang resin), 4-(2′,4′-dimethoxyphenylaminomethyl)-phenoxymethyl resin, 2,4-dimethoxybenzhydryl-amine resin, and 4-(2′,4′-dimethoxyphenyl-FMOC-amino-methyl)-phenoxyacetamidonorleucyl-MBHA resin (Rink amide MBHA resin). In addition, acid-sensitive resins also provide C-terminal acids, if desired. A particularly preferred protecting group for alpha amino acids is base-labile 9-fluorenylmethoxy-carbonyl (FMOC).

Suitable protecting groups for the side chain functionalities of amino acids chemically compatible with BOC (t-butyloxycarbonyl) and FMOC groups are well known in the art. When using FMOC chemistry, the following protected amino acid derivatives are preferred: FMOC-Cys(Trit), FMOC-Ser(But), FMOC-Asn(Trit), FMOC-Leu, FMOC-Thr(Trit), FMOC-Val, FMOC-Gly, FMOC-Lys(Boc), FMOC-Gln(Trit), FMOC-Glu(OBut), FMOC-His(Trit), FMOC-Tyr(But), FMOC-Arg(PMC (2,2,5,7,8-pentamethylchroman-6-sulfonyl)), FMOC-Arg(BOC)₂, FMOC-Pro, and FMOC-Trp(BOC). The amino acid residues can be coupled by using a variety of coupling agents and chemistries known in the art, such as direct coupling with DIC (diisopropyl-carbodiimide), DCC (dicyclohexylcarbodiimide), BOP (benzotriazolyl-N-oxytrisdimethylaminophosphonium hexa-fluorophosphate), PyBOP (benzotriazole-1-yl-oxy-tris-pyrrolidinophosphonium hexafluoro-phosphate), PyBrOP (bromo-tris-pyrrolidinophosphonium hexafluorophosphate); via performed symmetrical anhydrides; via active esters such as pentafluorophenyl esters; or via performed HOBt (1-hydroxybenzotriazole) active esters or by using FMOC-amino acid fluoride and chlorides or by using FMOC-amino acid-N-carboxy anhydrides. Activation with HBTU (2-(1H-benzotriazole-1-yl),1,1,3,3-tetramethyluronium hexafluorophosphate) or HATU (2-(1H-7-aza-benzotriazole-I -yl),1,1,3,3-tetramethyluronium hexafluoro-phosphate) in the presence of UOBt or HOAt (7-azahydroxybenztriazole) is preferred.

The solid phase method can be carried out manually, although automated synthesis on a commercially available peptide synthesizer (e.g., Applied Biosystems 431A or the like; Applied Biosystems, Foster City, Calif.) is preferred. In a typical synthesis, the first (C-terminal) amino acid is loaded on the chlorotrityl resin. Successive deprotection (with 20% piperidine/NMP (N-methylpyrrolidone)) and coupling cycles according to ABI FastMoc protocols (ABI user bulletins 32 and 33, Applied Biosystems are used to build the whole peptide sequence. Double and triple coupling, with capping by acetic anhydride, may also be used.

The synthetic mimetic peptide is cleaved from the resin and deprotected by treatment with TFA (trifluoroacetic acid) containing appropriate scavengers. Many such cleavage reagents, such as Reagent K (0.75 g crystalline phenol, 0.25 mL ethanedithiol, 0.5 mL thioanisole, 0.5 mL deionized water, 10 mL TFA) and others, can be used. The peptide is separated from the resin by filtration and isolated by ether precipitation. Further purification may be achieved by conventional methods, such as gel filtration and reverse phase HPLC (high performance liquid chromatography). Mimetics according to the present invention may be in the form of pharmaceutically acceptable salts, especially base-addition salts including salts of organic bases and inorganic bases. The base-addition salts of the acidic amino acid residues are prepared by treatment of the peptide with the appropriate base or inorganic base, according to procedures well known to those skilled in the art, or the desired salt may be obtained directly by lyophilization out of the appropriate base.

Generally, those skilled in the art will recognize that peptides as described herein may be modified by a variety of chemical techniques to produce compounds having essentially the same activity as the unmodified peptide, and optionally having other desirable properties. For example, carboxylic acid groups of the peptide may be provided in the form of a salt of a pharmaceutically-acceptable cation. Amino groups within the peptide may be in the form of a pharmaceutically-acceptable acid addition salt, such as the HCl, HBr, acetic, benzoic, toluene sulfonic, maleic, tartaric and other organic salts, or may be converted to an amide. Thiols can be protected with any one of a number of well-recognized protecting groups, such as acetamide groups. Those skilled in the art will also recognize methods for introducing cyclic structures into the peptides of this invention so that the native binding configuration will be more nearly approximated. For example, a carboxyl terminal or amino terminal cysteine residue can be added to the peptide, so that when oxidized the peptide will contain a disulfide bond, thereby generating a cyclic peptide. Other peptide cyclizing methods include the formation of thioethers and carboxyl- and amino-terminal amides and esters.

Specifically, a variety of techniques are available for constructing peptide derivatives and analogues with the same or similar desired biological activity as the corresponding peptide compound but with more favorable activity than the peptide with respect to solubility, stability, and susceptibility to hydrolysis and proteolysis. Such derivatives and analogues include peptides modified at the N-terminal amino group, the C-terminal carboxyl group, and/or changing one or more of the amido linkages in the peptide to a non-amido linkage. It will be understood that two or more such modifications can be coupled in one peptide mimetic structure (e.g., modification at the C-terminal carboxyl group and inclusion of a —CH₂— carbamate linkage between two amino acids in the peptide).

Amino terminus modifications include alkylating, acetylating, adding a carbobenzoyl group, and forming a succinimide group. Specifically, the N-terminal amino group can then be reacted to form an amide group of the formula RC(O)NH— where R is alkyl, preferably lower allcyl, and is added by reaction with an acid halide, RC(O)Cl or acid anhydride. Typically, the reaction can be conducted by contacting about equimolar or excess amounts (e.g., about 5 equivalents) of an acid halide to the peptide in an inert diluent (e.g., dichloromethane) preferably containing an excess (e.g., about 10 equivalents) of a tertiary amine, such as diisopropylethylamine, to scavenge the acid generated during reaction. Reaction conditions are otherwise conventional (e.g., room temperature for 30 minutes). Alkylation of the terminal amino to provide for a lower alkyl N-substitution followed by reaction with an acid halide as described above will provide for N-alkyl amide group of the formula RC(O)NR—. Alternatively, the amino terminus can be covalently linked to succinimide group by reaction with succinic anhydride. An approximately equimolar amount or an excess of succinic anhydride (e.g., about 5 equivalents) are used and the terminal amino group is converted to the succinimide by methods well known in the art including the use of an excess (e.g., ten equivalents) of a tertiary amine such as diisopropylethylamine in a suitable inert solvent (e.g., dichloromethane), as described in Wollenberg et al., U.S. Pat. No. 4,612,132, is incorporated herein by reference in its entirety. It will also be understood that the succinic group can be substituted with, for example, C₂— through C6— alkyl or —SR substiuents, which are prepared in a conventional manner to provide for substituted succinimide at the N-terminus of the peptide. Such alkyl substituents are prepared by reaction of a lower olefin (C₂— through C₆— alkyl) with maleic anhydride in the manner described by Wollenberg et al., supra., and —SR substituents are prepared by reaction of RSH with maleic anhydride where R is as defined above. In another advantageous embodiments, the amino terminus is derivatized to form a benzyloxycarbonyl-NH— or a substituted benzyloxycarbonyl-NH— group. This derivative is produced by reaction with approximately an equivalent amount or an excess of benzyloxycarbonyl chloride (CBZ-Cl) or a substituted CBZ-Cl in a suitable inert diluent (e.g., dichloromethane) preferably containing a tertiary amine to scavenge the acid generated during the reaction. In yet another derivative, the N-terminus comprises a sulfonamide group by reaction with an equivalent amount or an excess (e.g., 5 equivalents) of R—S(O)₂Cl in a suitable inert diluent (dichloromethane) to convert the terminal amine into a sulfonamide, where R is alkyl and preferably lower alkyl. Preferably, the inert diluent contains excess tertiary amine (e.g., ten equivalents) such as diisopropylethylamine, to scavenge the acid generated during reaction. Reaction conditions are otherwise conventional (e.g., room temperature for 30 minutes). Carbamate groups are produced at the amino terminus by reaction with an equivalent amount or an excess (e.g., 5 equivalents) of R—OC(O)Cl or R—OC(O)OC₆H₄-p-NO₂ in a suitable mert diluent (e.g., dichloromethane) to convert the terminal amine into a carbamate, where R is alkyl, preferably lower alkyl. Preferably, the inert diluent contains an excess (e.g., about 10 equivalents) of a tertiary amine, such as diisopropylethylamine, to scavenge any acid generated during reaction. Reaction conditions are otherwise conventional (e.g., room temperature for 30 minutes). Urea groups are formed at the amino terminus by reaction with an equivalent amount or an excess (e.g., 5 equivalents) of R—N═C═O in a suitable inert diluent (e.g., dichloromethane) to convert the terminal amine into a urea (i.e., RNHC(O)NH—) group where R is as defined above preferably, the inert diluent contains an excess (e.g., about 10 equivalents) of a tertiary amine, such as diusopropylethylamine. Reaction conditions are otherwise conventional (e.g., room temperature for about 30 minutes).

In preparing peptide mimetics wherein the C-terminal carboxyl group is replaced by an ester (e.g., —C(O)OR where R is alkyl and preferably lower allcyl), resins used to prepare the peptide acids are employed, and the side chain protected peptide is cleaved with base and the appropriate alcohol, e.g., methanol. Side chain protecting groups are then removed in the usual fashion by treatment with hydrogen fluoride to obtain the desired ester. In preparing peptide mimetics wherein the C-terminal carboxyl group is replaced by the amide —C(O)NR₃R₄, a benzhydrylamine resin is used as the solid support for peptide synthesis. Upon completion of the synthesis, hydrogen fluoride treatment to release the peptide from the support results directly in the free peptide amide (i.e., the C-terminus is —C(O)NH₂). Alternatively, use of the chloromethylated resin during peptide synthesis coupled with reaction with ammonia to cleave the side chain Protected peptide from the support yields the free peptide amide and reaction with an alkylamine or a dialkylamine yields a side chain protected alkylamide or dialkylamide (i.e., the C-terminus is —C(O)NRR₁, where R and R₁ are alkyl and preferably lower alkyl). Side chain protection is then removed in the usual fashion by treatment with hydrogen fluoride to give the free amides, allcylamides, or dialkylamides.

In another alternative embodiment, the C-terminal carboxyl group or a C-terminal ester can be induced to cyclize by displacement of the —OH or the ester (—OR) of the carboxyl group or ester respectively with the N-terminal amino group to form a cyclic peptide. For example, after synthesis and cleavage to give the peptide acid, the free acid is converted in solution to an activated ester by an appropriate carboxyl group activator such as dicyclohexylcarbodiimide (DCC), for example, in methylene chloride (CH₂Cl₂), dimethyl formamide (DMF), or mixtures thereof. The cyclic peptide is then formed by displacement of the activated ester with the N-terminal amine. Cyclization, rather than polymerization, can be enhanced by use of very dilute solutions according to methods well known in the art.

Peptide mimetics as understood in the art and provided by the invention are structurally similar to the paradigm peptides of the invention, but have one or more peptide linkages optionally replaced by a linkage selected from the group consisting of: —CH₂NH—, —CH₂S—, —CH₂CH₂—, —CH═CH— (in both cis and trans conformers), —COCH₂—, —CH(OH)CH₂—, and —CH₂SO—, by methods known in the art and further described in the following references: Spatola,1983, in CHEMISTRY AND BIOCHEMISTRY OF AMINO ACIDS, PEPTIDES, AND PROTEINS, (Weinstein, ed.), Marcel Dekker: New York, p. 267; Spatola, 1983, Peptide Backbone Modifications 1: 3; Morley, 1980, Trends Pharm. Sci. pp. 463-468; Hudson et al., 1979, Int. J. Pept. Prot. Res. 14: 177-185; Spatola et al., 1986, Life Sci. 38: 1243-1249; Hann, 1982, J. Chem. Soc. Perkin Trans. I 307-314; Almquist et al., 1980, J. Med. Chem. 23: 1392-1398; Jennings-White et al., 1982, Tetrahedron Lett. 23: 2533; Szelke et al., 1982, European Patent Application, Publication No. EP045665A; Holladay et al., 1983, Tetrahedron Lett. 24: 4401-4404; and Hruby, 1982, Life Sci. 31: 189-199, each of which is incorporated herein by reference. Such peptide mimetics may have significant advantages over polypeptide embodiments, including, for example: being more economical to produce, having greater chemical stability or enhanced pharmacological properties (such half-life, absorption, potency, efficacy, etc.), reduced antigenicity, and other properties.

Mimetic analogs of the tumor-inhibiting peptides of the invention may also be obtained using the principles of conventional or rational drug design (see, Andrews et al., 1990, Proc. Alfred Benzon Symp. 28: 145-165; McPherson, 1990, Eur. J. Biochem. 189:1-24; Hol et al., 1989a, in MOLECULAR RECOGNITION: CHEMICAL AND BIOCHEMICAL PROBLEMS, (Roberts, ed.); Royal Society of Chemistry; pp. 84-93; Hol, 1989b, Arzneim-Forsch. 39:1016-1018; Hol, 1986, Agnew Chem. Int. Ed. Engl. 25: 767-778, the disclosures of which are herein incorporated by reference).

In accordance with the methods of conventional drug design, the desired mimetic molecules are obtained by randomly testing molecules whose structures have an attribute in common with the structure of a “native” peptide. The quantitative contribution that results from a change in a particular group of a binding molecule can be determined by measuring the biological activity of the putative mimetic in comparison with the tumor-inhibiting activity of the peptide. In a preferred embodiment of rational drug design, the mimetic is designed to share an attribute of the most stable three-dimensional conformation of the peptide. Thus, for example, the mimetic may be designed to possess chemical groups that are oriented in a way sufficient to cause ionic, hydrophobic, or van der Waals interactions that are similar to those exhibited by the tumor-inhibiting peptides of the invention, as disclosed herein.

The preferred method for performing rational mimetic design employs a computer system capable of forming a representation of the three-dimensional structure of the peptide, such as those exemplified by Hol, 1989a, ibid.; Hol, 1989b, ibid.; and Hol, 1986, ibid. Molecular structures of the peptido-, organo- and chemical mimetics of the peptides of the invention are produced according to those with skill in the art using computer-assisted design programs commercially available in the art. Examples of such programs include SYBYL 6.5®, HQSAR™, and ALCHEMY 2000™ (Tripos); GALAXY™ and AM2000™ (AM Technologies, Inc., San Antonio, Tex.); CATALYST™ and CERIUS™ (Molecular Simulations, Inc., San Diego, Calif.); CACHE PRODUCTS™, TSAR™, AMBER™, and CHEM-X™ (Oxford Molecular Products, Oxford, Calif.)and CHEMBUILDER3D™ (Interactive Simulations, Inc., San Diego, Calif.).

The peptido-, organo- and chemical mimetics produced using the peptides disclosed herein using, for example, art-recognized molecular modeling programs are produced using conventional chemical synthetic techniques, most preferably designed to accommodate high throughput screening, including combinatorial chemistry methods. Combinatorial methods useful in the production of the peptido-, organo- and chemical mimetics of the invention include phage display arrays, solid-phase synthesis and combinatorial chemistry arrays, as provided, for example, by SIDDCO, Tuscon, Ariz.; Tripos, Inc.; Calbiochem/Novabiochem, San Diego, Calif.; Symyx Technologies, Inc., Santa Clara, Calif.; Medichem Research, Inc., Lemont, Ill.; Pharm-Eco Laboratories, Inc., Bethlehem, Pa.; or N.V. Organon, Oss, Netherlands. Combinatorial chemistry production of the peptido-, organo- and chemical mimetics of the invention are produced according to methods known in the art, including but not limited to techniques disclosed in Terrett, 1998, COMBINATORIAL CHEMISTRY, Oxford University Press, London; Gallop et al., 1994, “Applications of combinatorial technologies to drug discovery. 1. Background and peptide combinatorial libraries,” J. Med. Chem. 37: 1233-51; Gordon et al., 1994, “Applications of combinatorial technologies to drug discovery. 2. Combinatorial organic synthesis, library screening strategies, and future directions,” J. Med. Chem. 37: 1385-1401; Look et al., 1996, Bioorg. Med. Chem. Lett. 6: 707-12; Ruhland et al., 1996, J. Amer. Chem. Soc. 118: 253-4; Gordon et al., 1996, Acc.Chem. Res. 29: 144-54; Thompson & Ellman, 1996, Chem. Rev. 96: 555-600; Fruchtel & Jung, 1996, Angew. Chem. Int. Ed. Engl. 35: 17-42; Pavia, 1995, “The Chemical Generation of Molecular Diversity”, Network Science Center, www.netsci.org; Adnan et al., 1995, “Solid Support Combinatorial Chemistry in Lead Discovery and SAR Optimization,” Id., Davies and Briant, 1995, “Combinatorial Chemistry Library Design using Pharmacophore Diversity,” Id., Pavia, 1996, “Chemically Generated Screening Libraries: Present and Future,” Id.; and U.S. Pat. Nos. 5,880,972 to Horlbeck; 5,463,564 to Agrafiotis et al.; 5,331,573 to Balaji et al.; and 5,573,905 to Lerner et al.

In still another embodiment of the invention, inhibitors of HIV infection are identified by exposing a polypeptide encoded by a Target gene to a test compound; measuring the binding of the test compound to the polypeptide; and selecting a compound that binds to the polypeptide at a desired concentration, affinity, or avidity. In a preferred embodiment, the assay is performed under conditions conducive to promoting the interaction or binding of the compound to the polypeptide. One of skill in the art can determine such conditions based on the polypeptide and the compound being used in the assay.

In still another embodiment of the invention, a therapeutic compound of is identified by exposing an enzyme encoded by a Target gene to a test compound; measuring the activity of the enzyme encoded by the Target gene in the presence and absence of the compound; and selecting a compound that down-regulates the activity of the enzyme encoded by the Target gene. Methods to measure enzymatic activity are well known to those skilled in the art and are selected based on the identity of the enzyme being tested. For example, if the enzyme is a kinase phosphorylation assays can be used.

In addition to methods for identifying and producing a biological compound that inhibits HIV infection, the invention provides methods that down-regulate expression or function of a Target gene. For example, antisense RNA and DNA molecules may be used to directly block translation of mRNA encoded by these cellular genes by binding to targeted mRNA and preventing protein translation. Polydeoxyribonucleotides can form sequence-specific triple helices by hydrogen bonding to specific complementary sequences in duplexed DNA to effect specific down-regulation of target gene expression. Formation of specific triple helices may selectively inhibit the replication or expression of a Target gene by prohibiting the specific binding of functional trans-acting factors. The invention provides methods for identifying cellular targets for reduced gene expression or gene product activity, and methods for identifying said targets.

Ribozymes are enzymatic RNA molecules capable of catalyzing the specific cleavage of RNA. Ribozyme action involves sequence-specific hybridization of the ribozyme molecule to complementary target RNA, followed by endonucleolytic cleavage. Within the scope of the invention are ribozyme embodiments including engineered hammerhead motif ribozyme molecules that specifically and efficiently catalyze endonucleolytic cleavage of cellular RNA sequences, most preferably mRNA species. Antisense RNA molecules showing high-affinity-binding to target sequences can also be used as ribozymes by addition of enzymatically active sequences known to those skilled in the art.

Polynucleotides to be used in triplex helix formation should be single-stranded and composed of deoxynucleotides. The base composition of these polynucleotides must be designed to promote triple helix formation via Hoogsteen base pairing rules, which generally require sizeable stretches of either purines or pyrimidines to be present on one strand of a duplex. Polynucleotide sequences may be pyrimidine-based, which will result in TAT and CGC triplets across the three associated strands of the resulting triple helix. The pyrimidine-rich polynucleotides provide base complementarity to a purine-rich region of a single strand of the duplex in a parallel orientation to that strand. In addition, polynucleotides may be chosen that are purine-rich, for example, containing a stretch of G residues. These polynucleotides will form a triple helix with a DNA duplex that is rich in GC pairs, in which the majority of the purine residues are located on a single strand of the targeted duplex, resulting in GGC triplets across the three strands in the triplex.

Alternatively, sequences that can be targeted for triple helix formation can be increased by creating a so-called “switchback” polynucleotide. Switchback polynucleotides are synthesized in an alternating 5′-3′, 3′-5′ manner, so that they base pair with first one strand of a duplex and then the other, eliminating the necessity for a sizeable stretch of either purines or pyrimidines to be present on one strand of a duplex.

Both antisense RNA and DNA molecules, and ribozymes of the invention may be prepared by any method known in the art. These include techniques for chemically synthesizing polynucleotides well known in the art such as solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into host cells.

Various modifications to the nucleic acid molecules may be introduced as a means of increasing intracellular stability and half-life. Possible modifications include, but are not limited to, the addition of flanking sequences of ribonucleotides or deoxyribonucleotides to them5′ or 3′ ends of the molecule or the use of phosphorothioate or 2′ O-methyl rather than phosphodiesterase linkages within the oligodeoxyribonucleotide backbone.

Preferably, methods used to identify therapeutic compounds are customized for each Target gene or product. If the Target product is an enzyme, then the enzyme is expressed in cell culture and purified. The enzyme is then screened in vitro against therapeutic compounds to look for inhibition of that enzymatic activity. If the Target product is a non-catalytic protein, then it is expressed and purified, and therapeutic compounds tested for the ability to prevent, for example, the binding of a site-specific antibody or a Target-specific ligand to the Target product.

An assay of the present invention includes a viral entry assay. A cell line expressing CD4 can be used to determine in which step in the viral life cycle the block of replication occurs. Entry can be inhibited by blocking 1) the binding of the virus to the viral receptor (CD4), 2) binding to the co-receptor (CXCR-4 or CCR5), or 3) fusion of the virus and cell membranes.

In a preferred embodiment, therapeutic compounds that bind to Target products are identified, and those compounds are then further tested in a biological assay for inhibition of HIV infection. In preferred embodiments, the assay uses a multiplicity of cell samples, for example, arrayed in a 96-well plate format, using a cell line, most preferably a human cell line such as HeLa (human fibroblast) cell line. HIV infection assays may also be performed using primary T cells. In a preferred embodiment, the cell line expresses or has been modified to express the HIV cell surface receptor CD4, and more preferably also has been modified to express an expression vector that contains an HIV-1 LTR linked to the β-galactosidase gene. Using such cells, HIV inhibition can be monitored using β-galactosidase activity as the read-out of this assay. In this assay, HIV binds to CD4 on the cell surface and infects the cell. Upon infection with HIV, viral proteins including Tat are expressed. Tat binds to the HIV-1 LTR and promotes the expression of β-galactosidase. This expression of β-galactosidase can be detected and quantified. Inhibition of HIV replication by a compound would prevent or reduce expression of Tat and result in reduction of β-galactosidase expression compared to controls.

In certain embodiments of the invention, the therapeutic compound is not toxic to a human host cell that is not infected with HIV. In other embodiments, a therapeutic compound promotes apoptosis in a human host cell infected with HIV.

In certain embodiments the invention provides pharmaceutical compositions prepared from a therapeutically-effective amount of a therapeutic compound of the invention and a pharmaceutically-acceptable carrier, excipient or adjuvant. Pharmaceutically-acceptable carriers, excipients and adjuvants are well known to those with skill in the art. In another embodiment of the invention, resistance to HIV infection is conferred upon an individual by administering an effective amount of a pharmaceutical composition of the invention to the individual.

Preferred embodiments of the practice of the invention and its advantages over previously investigated detection methods are best understood by referring to Examples 1-4. The Examples, which follow, are illustrative of specific embodiments of the invention, and various uses thereof. They are set forth for explanatory purposes only, and are not to be taken as limiting the invention.

### EXAMPLE 1

### Preparation of Random Fragment Libraries for Isolating and Identifying Human Cell-Derived GSEs Exhibiting HIV Suppressive Activity

Three random fragment expression (RFE) libraries were constructed from mRNA isolated from HL-60 and HeLa cells, and from phytohemaglutinin (PHA) stimulated peripheral blood mononuclear cells (PBMCs).

A. HL60 RFE Library

The HL60 RFE library was prepared by isolating mRNA from uninduced HL60 cells (ATCC Ace. No. CCL 240) and then subtracting that mRNA with mRNA isolated from cells induced with TNF-α. This procedure represents a modification of that described by Coche et al. (1994, Nucleic Acids Res. 22:1322-23). Tracer mRNA was isolated from HL-60 cells transduced with the retroviral vector pLNCX (Miller and Rosman, 1989, BioTechniques 7:980-90) at different time points after induction with TNF-α (Boehringer Mannheim; Indianapolis, Ind.). The pLNCX sequences were used as an internal standard to monitor the enrichment of the sequences present in the tracer after subtraction.

Briefly, RNA was isolated from induced and uninduced cells using conventional methods (Sambrook et al., 1989, MOLECULAR CLONING: A LABORATORY MANUAL, 2^{nd} Ed., Cold Spring Harbor Laboratory Press, N.Y.). This RNA was annealed separately to oligo-dT magnetic beads (Dynal Biotech; Lake Success, N.Y.) and first strand cDNA was synthesized using reverse transcriptase and an oligo-dT primer. The RNA strand was then hydrolyzed and second strand cDNA synthesized from the induced cell first strand cDNA using a primer containing ATG codons in all three reading frames and an additional ten random nucleotides on the 3′ end. Single-stranded cDNA fragments were annealed to an excess of driver cDNA attached to the magnetic beads. This procedure was repeated several times until substantial enrichment in the pLNCX sequences was seen. The final population of single-stranded DNA (ssDNA) molecules was amplified using a primer containing TGA codons in all three reading frames and an additional ten random nucleotides on the 3′ end. The resulting population of cDNA fragments was then cloned into pLNCX. This step was taken to enrich for cellular sequences encoding products that might be important in supporting certain stages of the HIV life cycle in order to compensate for the low efficiency of retroviral transfer into OM10.1 cells. The HL60 library was found to comprise approximately 1 million transformants.

B. HeLa RFE Library

The HeLa RFE library was prepared using the method described by Gudkov et al. (1994, Proc. Natl. Acad. Sci. U.S.A. 91:3744). First, cDNA was prepared from HeLa cells and then partially digested with DNAse I in the presence of Mn⁺⁺ (Sambrook et al., 1989, Id.). Under these conditions, DNAse I is known to produce mostly double-stranded breaks. The resulting fragments were repaired using both the Klenow fragment of DNA polymerase I and T4 polymerase and then the fragments were ligated to synthetic double-stranded adaptors. The 5′ adaptor was prepared from the primers 5′-C-T-C-G-G-A-A-T-T-C-A-A-G-C-T-T-A-T-G-G-A-T-G-G-A-T-G-G-3′ (SEQ ID NO: 1) and 5′-C-A-T-C-C-A-T-C-C-A-T-A-A-G-C-T-T-G-A-A-T-T-C-C-3′ (SEQ ID NO: 2). The 3′ adaptor was prepared from the primers 5′-T-G-A-G-T-G-A-G-T-G-A-A-T-C-G-A-T-G-G-A-T-C-C-G-T-C-T-3′ (SEQ ID NO: 3) and 5′-T-C-C-T-A-G-A-C-G-G-A-T-C-C-A-T-C-G-A-T-T-C-A-C-T-C-A-C-T-C-A-3′ (SEQ ID NO: 4).

This randomly fragmented cDNA was then subjected to a normalization procedure to produce cDNA having a uniform abundance of different sequences in the population (Gudkov and Roninson, 1997, Methods in Molecular Biology 69:221, Humana Press, New York). This procedure was used to increase the probability of isolating GSEs from rare cDNAs, since total polyA⁺ RNA comprises a mixture of unequally represented sequences.

The randomly fragmented cDNA population was normalized by first denaturing 20 μg of cDNA by boiling for 5 minutes in 25 μL of TE buffer, followed by immediate cooling on ice. Then, 25 μL of 2×hybridization solution as described in Gudkov & Roninson was added, and the mixture was divided equally into four aliquots in Eppendorf tubes. One to two drops of mineral oil were added to each sample to avoid evaporation, and the tubes were placed into a 68° C. water bath for annealing. One tube was frozen every 12 hours. Following the last time-point, each of the annealing mixtures was diluted with water to a final volume of 500 μL and subjected to hydroxylapatite (HAP) chromatography. HAP suspension equilibrated with 0.01 M phosphate-buffered saline (PBS) was placed into Eppendorf tubes so that the volume of HAP pellet was approximately 100 μL. The tubes with HAP and all the solutions used below were preheated and kept at 65° C. Excess PBS was removed, and diluted annealing solution was added. After mixing by shaking in a 65° C. water bath, the tubes were left in the water bath until a HAP pellet was formed (a 15 second centrifugation was used to collect the pellet without exceeding 1000 g in the microcentrifuge to avoid damage of HAP crystals). The supernatant was carefully replaced with 1 mL of preheated 0.01 M phosphate buffered saline (PBS), and the process was repeated. To elute the ssDNA, the HAP pellet was suspended in 500 μL of PBS at the single-strand elution concentration determined (e.g., 0.16 M), the supernatant was collected, and the process was repeated. The supernatants were combined and traces of HAP were removed by centrifugation. The ssDNA was concentrated by centrifugation, and washed three times using 1 mL of water on a Centricon-100 column.

The isolated ssDNA sequences were amplified by polymerase chain reaction (PCR) using sense primers from each adapter and a minimal number of cycles to obtain 10 μg of the product. The size of the PCR product that remained within the desired range (200-500 bp) was ascertained. The normalization quality was tested by Southern or slot-blot hybridization with ³²P-labeled probes for high, moderate- and low-expressing genes using 0.3-1.0 μg of normalized cDNA/lane. β-actin and β-tubulin cDNAs were used as probes for high-expressing (high abundance) genes, c-myc and topoisomerase II cDNAs were used as probes for moderate-expressing genes, and c-fos cDNA was used as a probe for low-expressing (low abundance) genes. The cDNAs isolated after different annealing times were compared with the original unnormalized cDNA. The probes were ensured to have a similar size and specific activity. The best-normalized ssDNA fraction (i.e., the population which produced the most uniform signal intensity with different probes) was used for large-scale PCR amplification to synthesize at least 20 μg of the product for cloning. More ssDNA template was used to obtain the desired amount by scaling up the number of PCR cycles or the reaction volume.

Following normalization, the mixture of randomly fragmented cDNA was digested with BamHI and EcoRI, column purified, and then ligated into either pLNCX or pLNGFRM (pLNGFRM differs from pLNCX in that the neo gene has been replaced with a truncated low affinity nerve growth factor receptor (NGFR) gene). Cells transduced with pLNGFRM express a truncated receptor on their surface that can be easily selected by an anti-NGFR antibody by, inter alia, fluorescence activated cell sorting (FACS). The ligation mixture was introduced into E. coli, and approximately 100,000 transformants were obtained. The size distribution of the cloned fragments was analyzed by PCR using primers derived from vector sequences adjacent to the adapter sequences.

C. PBMC RFE Library

The PBMC RFE library was prepared by isolating the mononuclear (buffy coat) fraction of whole blood from four healthy donors, from which peripheral blood mononuclear cells (PBMCs) were purified by Ficoll gradient centrifugation followed by stimulation with PHA (1 μg/mL). Cells were removed at 5, 10, and 24 hours following the addition of PHA and total RNA was isolated by Trizol extraction. The isolated total RNA collected from the four donors was then pooled, yielding three populations corresponding to the time at which the cells were removed following PHA treatment. Poly-A⁺ mRNA was purified from the total RNA using the Gibco Superscript Choice system for cDNA synthesis (Gibco BRL; Bethesda, Md.) and a random primer. The cDNA was normalized using the PCR-Select cDNA Subtraction kit (Clontech; Palo Alto, Calif.), based on the suppression subtractive hybridization methods of Diatchenko et al. (1996, Proc. Natl. Acad. Sci. U.S.A. 93:6025-30), and the primers 5′-T-A-G-G-G-C-T-C-G-A-G-C-C-G-C-C-A-C-C-A-T-G-3′ (SEQ ID NO: 5) and 5′-A-T-C-C-C-T-G-C-A-G-G-T-C-A-C-T-C-A-C-T-C-A-3′ (SEQ ID NO: 6). The normalized random fragments were digested with XhoI and SseI, purified on quick spin columns (Qiagen; Valencia, Calif.) and ligated into the SseI and XhoI sites of a bicistronic retroviral vector, pLXEMCVNgfr. This vector is based on pLXSNgfr. Modifications included the replacement of the SV40 promoter with encephalomyocarditis virus (EMVC) internal ribosomal entry site (IRES) isolated from the plasmid pCITE (Amersham Biosciences; Piscataway, N.J.). The ligation mixture was introduced into competent cells, and approximately 50 million transformants were obtained.

### EXAMPLE 2

### Transduction and Selection of Human Cell-Derived GSEs

HL-60 RFE libraries prepared as described in Example 1 were introduced into a packaging cell line, PA317 (ATCC Acc. No. CRL 9078), and converted into retrovirus for infection of OM10.1 cells (ATCC Acc. No. CRL 10850; U.S. Pat. No. 5,256,534). OM10.1 cells transduced with a pLNCX-based HL-60 RFE library were co-cultured and selected with G418. OM 10.1 cells transduced with a pLNGFRM-based or pLXEMCVNgfr-based HL-60 RFE library were first subjected to spinoculation (centrifugation of target cells at 1200×g for 90 minutes in the presence of filtered retroviral supernatant) and then selected by FACS sorting of the NGFR⁺ population. Following selection, OM10.1 cells harboring the entire RFE library were induced with 10 U/mL of TNF-α at 37° C. for 24 hours, stained with antibody, and then sorted for CD4 expression. Genomic DNA from the CD4⁺ cells was purified and used for PCR amplification of inserts using vector-derived primers. The amplified mixture was digested with EcoRI and BamHI and cloned back into the retroviral vector. This selection was repeated for additional rounds.

Normalized RFE libraries prepared from HeLa cells or PBMCs as described in Example 1 were transferred into CEM-ss cells (Cat. No. 776; NIH AIDS Research and Reference Reagent Program) and neo resistant and NGFR⁺ populations were isolated. The HeLa and PMBC RFE libraries each comprised 50×10⁶ independent recombinant clones. Following introduction of the RFE libraries into CEM-ss cells, the CEM-ss cells were infected with a TCID₅₀ of 3000/10⁶ cells of HIV-1_{IIIB} (Cat. No. 398; NIH AIDS Research and Reference Reagent Program). Because it has been suggested that syncytia formation can be prevented by blocking the interaction between gp120 expressed on the surface of an infected cells and CD4 on the surface of an uninfected cells, 3 μg/mL of a purified anti-CD4 monoclonal antibody, L77 (Becton Dickinson), was added at 4 and 7 days following infection. The L77 antibody does not prevent HIV infection of a cell. At 8-10 days after infection, a subpopulation of CD4⁺/p24⁻ cells corresponding to the uninfected cells was sorted. Genomic DNA from the isolated CD4⁺/p24⁻ cells was purified and used for PCR amplification of inserts with the vector-derived primers. The amplified mixture was digested with EcoRI and BamHI and then cloned back into the retroviral vector. This selection was repeated for additional rounds.

These results demonstrated that each of the cell mRNA-derived RFE libraries contained species that inhibited HIV infection by reducing expression of a cellular gene or activity of a cellular gene product that was expressed in the uninfected cells from which each RFE library was prepared.

### EXAMPLE 3

### Recovery and Sequencing of Human Cell-Derived GSEs

HIV infection inhibiting human GSEs were obtained from the uninfected cell populations described in Example 2 as follows. Genomic DNA was isolated from the HIV infection-resistant selected OM10.1 or CEM-ss cells prepared as described in Example 2 by first centrifuging the selected cells, resuspending the cell pellet in a solution of 0.1% Triton X-100, 20 μg/mL proteinase K, and 1×PCR buffer, incubating the cells at 55° C. for 1 hour, and then boiling the cell suspension for 10 minutes. Genomic DNA was used for PCR amplification using vector-derived primers to produce fragments comprising the GSE inserts, which were then cloned into the retroviral vector, and introduced into E. coli using standard transformation techniques. Individual plasmids were purified from E. coli clones using QIAGEN plasmid purification kits. Inserts were sequenced by the dideoxy procedure (using the AutoRead Sequencing Kit, Pharmacia Biotech or the Prism Big Dye Terminator Cycle Sequencing Ready Reaction Kit, ABI) and analyzed on a Pharmacia LKB A.L.F. or ABI 3700 DNA sequencer. Sequences were analyzed using the DNASTAR program or other proprietary data mining procedure.

As described in Example 2, two independent selection strategies were performed on two different cell lines (OM10.1 and CEM-ss) into which three independent RFE libraries were introduced. The GSEs identified using these selection strategies, and the human cellular genes from which these GSEs were derived, are indicated in Tables 1A and 1B, respectively. The Tables set forth the identities of each of the genes from which GSEs were produce, whether each GSE was sense or antisense in orientation, and the portions of these genes that comprised the GSE, with reference to the nucleotide sequence.

### EXAMPLE 4

### Cell Population Sorting Based on p24 Expression Using Immunofluorescence and Flow Cytometry

Since intracellular p24 accumulation and surface CD4 down-modulation are associated with HIV-1 replication, successful interference with HIV-1 infection should result in an enrichment in cells displaying a p24⁻/CD4⁺ phenotype. Cells possessing this phenotype (i.e., uninfected cells) were identified by immunofluorescence 8-10 days following challenge with HIV. First, CD4⁺ cells were isolated from the challenged cell population (1×10⁷ cells) by washing the cells twice with Assay Buffer (500 mL PBS, 1 mL of 0.5 mM of EDTA, pH 8, 0.5 mL of 10% sodium azide, and 10 mL of fetal bovine serum), and then resuspending the cells in 500 μL PBS containing 50 μL of anti-CD4 antibody (Q4120 PE; Sigma). Following incubation at 4° C. for 30 minutes, 5 mL of Assay Buffer was added and the cells were centrifuged at 1200 rpm for 4 minutes. The cells were then washed twice with Assay Buffer and CD4⁺ cells sorted from the population by FACS. The aforementioned procedure was performed under sterile conditions.

To identify cells within the CD4⁺ population that do not express p24, the sorted cell population (1×10⁶ cells) was washed twice with Assay Buffer, and then suspended in 100 μL of Assay Buffer and 2 mL of Ortho PermeaFix Solution (Ortho Diagnostics). The cells were incubated at room temperature for 40 minutes, centrifuged at 1200 rpm and 4° C. for 4 minutes, and then resuspended in 2 mL Wash Buffer (500 mL PBS, 25 mL fetal bovine serum, 1.5% bovine serum albumin and 0.0055% EDTA). Following centrifugation at room temperature for 10 minutes, the cells were resuspended in 50 μL Wash Buffer diluted 1:500 with IgG₂ₐ antibody, and incubated at 4° C. for 20 minutes. Following this incubation, 5-10 μL of anti-p24 antibody (KC57-FITC; Coulter) was added and the cells were incubated at 4° C. for 30 minutes. The cells were then washed twice with Wash Buffer and analyzed by flow cytometry. These assays indicated that the GSEs isolated according to the methods described in Examples 2 and 3 inhibited HIV infection in these cells as assayed by the absence of p24 expression.

It should be understood that the foregoing disclosure emphasizes certain specific embodiments of the invention and that all modifications or alternatives equivalent thereto are within the spirit and scope of the invention as set forth in the appended claims.

TABLE IA
GSEs and Human Cellular Genes Involved in Inhibition of HIV Infection

| | | Accession | |
|---|---|---|---|
| LHL SELECTION | | No. | COORDINATES |
| NADH Dehydrogenase URF6 | AS | V00662 | 14129-14263 |
| NADH Dehydrogenase URF 2 | AS | V00662 | 4745-4860 |
| Squalene synthetase | S | X69141 | 1466-1586 |
| RTLV associated endogenous | S | M18048 | 2929-3028 |
| retrovirus | | | |
| Human 2-oxoglutarate de- | S | D10523 | 2837-2943 |
| hydrogenase | | | |
| Human type 2 pyruvate | S | M26252 | 945-1049 |
| kinase/cytosolic | | | |
| thyroid hormone binding protein | | | 922-1027 |
| (TCBA) | | | |
| Human calnexin | AS | L10284 | 1045-1220 |
| Human ubiquitin specific protease | S | Z72499 | 974-1087 |
| (HAUSP) | | | |
| Human ADP ribosylation factor 3 | S | M74491 | 351-471 |
| (ARF3) | | | |
| Human initiation factor 4B (eIF4B) | AS | X55733 | 1061-1157 |
| Human translation initiation factor | AS | U78525 | 2321-2430 |
| (eIF3) | | | |
| Glucosidase II (3′UTR) | S | D42041 | 2909-3047 |
| Glucosidase II | AS | AJ000332 | 2286-2402 |
| Na⁺-D-glucose cotransport regula- | AS | X82877 | 5744-5835 |
| tor gene | | | |
| Integrin associated protein (CD47) | AS | Z25521 | 283-375 |
| CD44 | AS | X55150 | 8-75 |
| BDP-1 tyrosine phosphatase | S | X79568 | 1979-2053 |
| Phosphatidyl inositol kinase | AS | Z46973 | 67-124 |
| (P13K) | | | |
| Elongation factor 1 (EF-1) | AS | X03558 | 5-128 |
| Mitochondrial aspartate amino | AS | M22632 | 1-57 |
| transferase | | | |
| Double strand break repair gene | S | X98294 | 774-852 |
| Rat guanine nucleotide releasing | S | X70496 | 574-627 |
| protein | | | |
| Antiproliferative factor | S | X61123 | 411-487 |
| BTG-1 | | | |
| Lymphocyte specific protein 1 | S | M33552 | 4-46 |
| Protein phosphatase 2A (α | AS | M64929 | 1656-1744 |
| regulatory) | | | |
| Eukaryotic release factor 1 | S | U90176 | 805-921 |
| (ERF-1) | | | |
| GTP binding protein | S | L10665 | 925-981 |
| Importin beta subunit | AS | L38951 | 3042-3176 |
| Cell adhesion molecule L1 | AS | M77640 | 3125-3213 |
| (L1CAM) | | | |
| Heparan sulfate proteoglycan | AS | J04621 | 2845-2947 |
| (HSPG) | | | |
| Zinc finger factor 1 | AS | U48809 | 4227-4359 |
| Bone morphogenic protein (BMP1- | S | M22488 | 978-1050 |
| 6) | | | |
| U-snRNP associated cyclophilin | AS | AF016371 | 603-781 |
| Recepin (endoprotease) | AS | U03644 | 294-389 |
| Lipocortin II/ Annexin II | S | D00017 | 736-861 |
| hnRNP A1 | S | X12671 | 4712-4816 |
| ArgBP2a (Arg/Ab1 interacting | S | AF049884 | 1392-1465 |
| protein) | | | |
| Keratin related protein (IFN-γ | AS | X62571 | 1088-1228 |
| regulated) | | | |
| GLUCOSYLTRANSFERASE | S | AJ224875 | 853-946 |
| Rox (transcriptional repressor) | AS | X96401 | 4621-4717 |
| p18 protein | S | J04991 | 556-645 |
| E1c (small nucleolar RNA) | S | U12211 | 17-123 |
| Ferritin heavy subunit | AS | M12937 | 290-385 |
| p40 (7-transmembrane protein) | AS | Y11395 | 1168-1286 |
| | | Accession | |
| H1C SELECTION | | No. | Coordinates |
| MIP-1α | AS | M23458 | 9-50 |
| | | | 11-50 |
| | | | 10-50 |
| | | | 12-50 |
| HSP90 | AS | M16660 | 2175-2214 |
| | | | 2133-2214 |
| | | | 2152-2214 |
| | | | 2054-2090 |
| MTP-1β | AS | D90145 | 1118-1154 |
| | | | 1119-1158 |
| Human type 2 pyruvate kinase/ | S | M26252 | 2020-2164 |
| cytosolic thyroid hormone | | | |
| binding protein (TCBA) | | | |
| NF-kB binding subunit | AS | M58603 | 3382-3433 |
| | | | 3285-3439 |
| | | | 3376-3439 |
| BBC-1 | AS | X64707 | 527-648 |
| | | | 554-648 |
| α-enolase | AS | AF035286 | 927-1084 |
| Translationally controlled tumor | AS | L13806 | 74-158 |
| protein (TCTP) | | | |
| DAP 5 (eIF4G homolog) | AS | X89713 | 389-505 |
| FK-506 binding protein 1A | AS | M34539 | 1154-1473 |
| TRAP-beta | AS | D37991 | 463-567 |
| TID1 (tumorous imaginal disc | S | AF061749 | 2391-2480 |
| homolog) | | | |
| Heparin binding protein (HIP) | AS | U49083 | 2-105 |
| | | | 1-83 |
| Poly A binding protein (PABP) | AS | U68103 | 147-198 |
| Cytokine effector-inflammatory | AS | X52147 | 2-38 |
| response | | | |
| Nuclear U4A RNA | AS | V00592 | 29-88 |
| hnRNP A2/B1 | AS | D28877 | 2442-2477 |
| IL-1 beta | AS | K027701 | 673-830 |
| TNF-cat receptor | AS | S63368 | 2346-2399 |
| HYPK mRNA | S | AF049613 | 292-364 |
| HIV-1 TAR binding protein | S | L22453 | 296-355 |
| TRAP-delta | AS | Z69043 | 236-281 |
| ATP6E | AS | NM001696 | 1-37 |
| M025 | AS | AF113536 | 163-264 |
| CD69 | S | Z22576 | 23-141 |
| Mitochondrial cytochrome | AS | M12548 | 5912-6085 |
| oxidase I | | | |
| Csa-19 | AS | U12404 | 327-499 |
| NOVEL GENE | S | M73791 | 341-497 |
| 14-3-3 zeta protein | S | U28964 | 368-452 |
| Nef interacting protein (Nip 7-1) | S | U83843 | 583-665 |
| EF-1 delta | AS | Z21507 | 906-977 |
| E16 mRNA | AS | AF077866 | 799-882 |
| Arginyl tRNA synthetase | AS | NM_{—} | 15-236 |
| | | 002877.1 | |
| Novel nuclear targeted gene | AS | AB015345 | 119-224 |
| eIF4AII | AS | D30655 | 1-97 |
| WBSCRI | S | AF045555 | 25022-25062 |
| C2IORF4 | S | NM_006134 | 447-579 |
| | S | | 447-579 |
| Protein phosphatase 2A B56 | AS | NM_002719 | 1001-1224 |
| gamma 1 | | | |
| DAP12 | AS | AF019563 | 3680-3875 |
| Programmed cell death 4 (PDCD4) | AS | NM_014456 | 246-374 |
| Glutaredoxin | AS | U40574 | 1098-1181 |
| | AS | | 1098-1181 |
| eIF4AI | S | NM_001416 | 348-430 |
| GA17 | AS | NM_006360 | 17-57 |
| | AS | | 17-57 |
| MAD-3/NFKBIA | AS | NM_020529 | 378-536 |
| RANTES | S | NM_002985 | 226-380 |
| IL-6 | AS | AF048692 | 606-664 |
| FYN binding protein | AS | AF001862 | 5-211 |
| ABC transporter | AS | AJ005016 | 62-152 |
| hSHIP | AS | NM_005541 | 2700-2767 |
| IEX-IL | AS | AF071596 | 356-446 |
| CDC42 | AS | NM_001791 | 399-673 |
| Tryptophanyl tRNA synthetase | AS | NM_004184 | 1220-1273 |
| TRAP-gamma | S | NM_007107 | 69-104 |
| CXCR-4 | AS | NM_003467 | 14-214 |
| Cyclin T1 | AS | NM_001240 | 15-86 |
| PDIR | AS | NM_006810 | 1130-1180 |
| G3PDH | AS | NM_002046 | 508-624 |
| CCR4 | AS | NM_005508 | 1505-1566 |
| Guanine nucleotide binding protein | AS | NM_006098 | 101-198 |
| (GNB2L1) | | | 121-198 |
| Cathepsin B | AS | NM_001908 | 1276-1401 |
| Cathepsin L | AS | NM_001912 | 1242-1314 |
| Vacuolar H+ ATPase proton | AS | NM_001694 | 1063-1160 |
| channel subunit 6C | | | |
| Prolyl 4-hydroxylase | AS | NM_000918 | 1603-1529 |
| Protein phosphatase 2A α catalytic | S | NM_002715 | 854-997 |
| ATP1A1 | AS | NM_000701 | 86-243 |
| O-linked GlcNAc transferase | S | NM_003605 | 4526-4761 |
| CDP-diacylglycerol synthase 2 | S | AF069532 | 654-757 |
| FoF1 ATP synthase f subunit | AS | NM_004889 | 209-279 |
| Guanylate binding protein | AS | NM_002053 | 711-815 |
| ATP5G2 | AS | NM_005176 | 12-206 |
| Phosphorylase kinase, alpha 2 | AS | NM_000292 | 1917-2092 |
| SOD-2 | AS | X65965 | 4987-5090 |
| NADH ubiquinine oxidoreductase | AS | NM_005005 | 83-157 |
| B22 subunit | | | |
| DEAD/H Box 5 | S | AF015812 | 5364-5419 |
| DEAD/H 9 (Nuclear DNA helicase | AS | NM_001357 | 3100-3227 |
| II) | | | |
| Aryl Sulfotransferase | AS | U20499 | 1136-1204 |
| Cytochrome b gene | AS | AF254896 | 129-324 |
| 5-aminoimidazole-4-carboxamide | AS | NM_004044 | 361-415 |
| ribonucleotide formyltransferase/ | | | |
| IMP cyclohydrolase (ATIC) | | | |
| Cytochrome bc-1 core protein | AS | NM_003366 | 28-82 |
| Integrin, alpha X (CD11c) | AS | NM_000887 | 4019-4095 |
| Long chain polyunsturated fatty | AS | NM_021814 | 4-52 |
| acid elongation enzyme (HELO1) | | | |
| Nucleophosmin-retinoic acid | AS | U41743 | 1-167 |
| receptor alpha fusion protein NPM- | | | |
| RAR | | | |
| Protein phosphatase I regulatory | S | NM_021959 | 732-797 |
| Aldehydedehydrogenase | AS | NM_000692 | 981-1005 |
| Glucosamine-6-phosphate | S | AF048826 | 239-332 |
| deaminase | | | |
| DDX3 | S | NM_001356 | 2013-2173 |
| ATP synthase epsilon chain | AS | NM_006886 | 34-100 |
| (ATP5E) | | | |
| Calpain, small subunit (CAPNS1) | S | NM_001749 | 295-500 |
| Coactivator-associated arginine | AS | XM_032719 | 922-1101 |
| methyltransferase- 1 (CARM1) | | | |
| Casein kinase 1, epsilon | S | NM_001893 | 996-1162 |
| (CSNK1E) | | | |
| Cathepsin D (CTSD) | S | NM_001909 | 1080-1319 |
| CCR7 | AS | NM_001838 | 1214-1389 |
| CD68 | AS | NM_001251 | 234-410 |
| CD74 | AS | NM_004355 | 18-238 |
| CDC-like kinase 3 (CLK3) | S | NM_001292 | 313-424 |
| Cysteine sulfinic acid de- | AS | NM_015989 | 676-860 |
| carboxylase-related protein | | | |
| (CSAD) | | | |
| Colony stimulating factor 3 | AS | NM_000760 | 55-234 |
| receptor (CSF3R) | | | |
| Casein kinase 1, gamma 2 | AS | NM_001319 | 87-349 |
| (CSNK1G2) | | | |
| RNA helicase, DECD variant | AS | NM_005804 | 1229-1447 |
| (DDXL) | | | |
| DNA cytosine-5-methyltransferase | AS | NM_022552 | 329-588 |
| 3 alpha (DNMT3A) | | | |
| Dual specificity phosphatase 1 | AS | NM_004417 | 17-245 |
| (DUSP1) | | | |
| G protein-coupled receptor kinase | AS | NM_002082 | 532-765 |
| 6 (GPRK6) | | | |
| Human ADP/ATP translocase | AS | J03592 | 469-658 |
| Leukocyte receptor cluster member | S | XM_044313 | 1182-1357 |
| 8 (LENG8) | | | |
| Mitogen-activated protein kinase 7 | S | NM_005043 | 698-853 |
| (MAP2K7) | | | |
| Macrophage migration inhibitory | AS | NM_002415 | 49-323 |
| factor (MIF) | | | |
| Misshapen/NIK-related kinase | AS | NM_015716 | 981-1110 |
| (MINK) | | | |
| Protein expressed in non-metastatic | AS | NM_005009 | 449-646 |
| cells 4 (NME4) | | | |
| Nonreceptor protein-tyrosine | AS | M63877 | 2717-2880 |
| kinase (fgr) | | | |
| P101-P13K | AS | NM_014308 | 85-286 |
| P2X1 receptor gene | AS | AF078925 | 16-45 |
| Phosphodiesterase 3B (PDE3B) | S | NM_000922 | 12-170 |
| Protein tyrosine kinase 2 beta | AS | NM_004103 | 313-546 |
| (PTK2B) | | | |
| Protein tyrosine phosphatase HD- | S | AB02519422 | 18-2383 |
| PTP (PTPN23) | | | |
| RAB7 | S | X93499 | 447-584 |
| SLC11A1 | AS | NM_000578 | 1163-1399 |
| PI-3-kinase-related (SMG1) | AS | XM₁₃ | 4357-4627 |
| | | 043965 | |
| Serine/threonine kinase 10 | AS | NM_005990 | 760-900 |
| (STK10) | | | |
| TAP1 | AS | NM_000593 | 1898-2095 |
| Thromboxane A2 receptor | AS | L14561 | 2404-2580 |
| (TBXA2R) | | | |
| Tyrosine kinase 2 (TYK2) | S | NM_003331 | 18-261 |
| Ubiquitin-conjugating enzyme | AS | NM_003969 | 66-225 |
| E2M (UBE2M) | | | |
| Uridine phosphorylase (UP) | AS | NM_003364 | 1090-1343 |
| Gamma-aminobutyric acid B | S | NM_021905 | 898-1091 |
| receptor 1 (GABBR1) | | | |

AS = Antisense
S = Sense

TABLE IB
GSEs and Human Cellular Genes Involved in Inhibition of HIV Infection

| | | Accession | |
|---|---|---|---|
| H1C SELECTION | | No. | Coordinates |
| MIP-1α | AS | M23458 | 9-50 |
| | | | 11-50 |
| | | | 10-50 |
| | | | 12-50 |
| HSP90 | AS | M16660 | 2175-2214 |
| | | | 2133-2214 |
| | | | 2152-2214 |
| | | | 2054-2090 |
| MIP-1β | AS | D90145 | 1118-1154 |
| | | | 1119-1158 |
| Human type 2 pyruvate kinase/cyto- | S | M26252 | 2020-2164 |
| solic thyroid hormone binding protein | | | |
| (TCBA) | | | |
| NF-kB binding subunit | AS | M58603 | 3382-3433 |
| | | | 3285-3439 |
| | | | 3376-3439 |
| BBC-1 | AS | X64707 | 527-648 |
| | | | 554-648 |
| α-enolase | AS | AF035286 | 927-1084 |
| Translationally controlled tumor pro- | AS | L13806 | 74-158 |
| tein (TCTP) | | | |
| DAP 5 (eIF4G homolog) | AS | X89713 | 389-505 |
| FK-506 binding protein 1A | AS | M34539 | 1154-1473 |
| TRAP-beta | AS | D37991 | 463-567 |
| TID1 (tumurous imaginal disc homo- | S | AF061749 | 2391-2480 |
| log) | | | |
| Heparin binding protein (HIP) | AS | U49083 | 2-105 |
| | | | 1-83 |
| Poly A binding protein (PABP) | AS | U68103 | 147-198 |
| Cytokine effector-inflammatory re- | AS | X52147 | 2-38 |
| sponse | | | |
| Nuclear U4A RNA | AS | V00592 | 29-88 |
| hnRNP A2/B1 | AS | D28877 | 2442-2477 |
| IL-1 beta | AS | K027701 | 673-830 |
| TNF-αreceptor | AS | S63368 | 2346-2399 |
| HYPK mRNA | S | AF049613 | 292-364 |
| HIV-1 TAR binding protein | S | L22453 | 296-355 |
| TRAP-delta | AS | Z69043 | 236-281 |
| ATP6E | AS | NM_001696 | 1-37 |
| MO25 | AS | AF113536 | 163-264 |
| CD69 | S | Z22576 | 23-141 |
| Mitochondiral cytochrome oxidase I | AS | M12548 | 5912-6085 |
| Cas-19 | AS | I12404 | 327-499 |
| NOVEL GENE | S | M73791 | 341-497 |
| 14-3-3 zeta protein | S | U28964 | 368-452 |
| Nef interacting protein (Nip 7-1) | S | U83843 | 583-665 |
| EF-1 delta | AS | Z21507 | 906-977 |
| E16 mRNA | AS | AF077866 | 799-882 |
| Arginyl tRNA synthetase | AS | NM002877.1 | 15-236 |
| Novel nuclear targeted gene | AS | AB015345 | 119-224 |
| eIF4AII | AS | D30655 | 1-97 |
| WBSCRI | S | AF045555 | 25022-25062 |
| C21ORF4 | S | NM_006134 | 447-579 |
| | S | | 447-579 |
| Protein phosphatase 2A B56 gamma | AS | NM_002719 | 1001-1224 |
| 1 | | | |
| DAP12 | AS | AF019563 | 3680-3875 |
| Programmed cell death 4 (PDCD4) | AS | NM_014456 | 246-374 |
| Glutaredoxin | AS | U40574 | 1098-1181 |
| | AS | | 1098-1181 |
| eIF4AI | S | NM_001416 | 348-430 |
| GA17 | AS | NM_006360 | 17-57 |
| | AS | | 17-57 |
| MAD-3/NFKBIA | AS | NM_020529 | 378-536 |
| RANTES | S | NM_002985 | 226-380 |
| IL-6 | AS | AF048692 | 606-664 |
| FYN binding protein | AS | AF001862 | 5-211 |
| ABC transporter | AS | AJ005016 | 62-152 |
| hSHIP | AS | NM_005541 | 2700-2767 |
| IEX-IL | AS | AF071596 | 356-446 |
| CDC42 | AS | NM_001791 | 399-673 |
| Tryptophanyl tRNA synthetase | AS | NM_004184 | 1220-1273 |
| TRAP-gamma | S | NM_007107 | 69-104 |
| CXCR-4 | AS | NM_003467 | 14-214 |
| Cyclin T1 | AS | NM_001240 | 15-86 |
| PDIR | AS | NM_006810 | 1130-1180 |
| G3PDH | AS | NM_002046 | 508-624 |
| CCR4 | AS | NM_005508 | 1505-1566 |
| Guanine nucleotide binding protein | AS | NM_006098 | 101-198 |
| (GNB2L1) | | | 121-198 |
| Cathepsin B | AS | NM_001908 | 1276-1401 |
| Cathepsin L | AS | NM_001912 | 1242-1314 |
| Vacuolar H+ ATPase proton channel | AS | NM_001694 | 1063-1160 |
| subunit 6C | | | |
| Prolyl 4-hydroxylase | AS | NM_000918 | 1603-1529 |
| Protein phosphatase 2A α catalytic | S | NM_002715 | 854-997 |
| ATP1A1 | AS | NM_000701 | 86-243 |
| O-linked GlcNAc transferase | S | NM_003605 | 4526-4761 |
| CDP-diacylglycerol synthase 2 | S | AF069532 | 654-757 |
| FoF1 ATP Synthetase f subunit | AS | NM_004889 | 209-279 |
| Guanylate binding protein | AS | NM_002053 | 711-815 |
| ATP5G2 | AS | NM_005176 | 12-206 |
| Phosphorylase kinase, alpha 2 | AS | NM_000292 | 1917-2092 |
| SOD-2 | AS | X65965 | 4987-5090 |
| NADH ubiquinone oxireductase B22 | AS | NM_005005 | 83-157 |
| subunit | | | |
| DEAD/H Box 5 | S | AF015812 | 5364-5419 |
| DEAD/H 9 (Nuclear DNA Helicase | AS | NM_001357 | 3100-3227 |
| II) | | | |
| Aryl Sulfotransferase | AS | U20499 | 1136-1204 |
| Cytochrome b gene | AS | AF254896 | 129-324 |
| 5-aminoimidazole-4-carboxamide | AS | NM_004044 | 361-415 |
| ribonucleotide formyltransferase/IMP | | | |
| cyclohydrolase (ATIC) | | | |
| Cytochrome bc-1 core protein | AS | NM_003366 | 28-82 |
| Integrin, alpha X (CD11c) | AS | NM_00887 | 4019-4095 |
| Long chain polyunsaturated fatty acid | AS | NM_021814 | 4-52 |
| elongation enzyme (HELO1) | | | |
| Nucleophosmin-retinoic acid receptor | AS | U41743 | 1-167 |
| alpha fusion protein NPM-RAR | | | |
| Protein phosphatase I regulatory | S | NM_021959 | 732-797 |
| Aldehyde dehydrogenase | AS | NM_000692 | 981-1005 |
| Glucosamine-6-phosphate deaminase | S | AF048826 | 239-332 |
| DDX3 | S | NM_001356 | 2013-2173 |
| ATP synthetase epsilon chain | AS | NM_006886 | 34-100 |
| (ATP5E) | | | |
| Calpain, small subunit (CAPNS1) | S | NM_001749 | 295-500 |
| Coactivator-associated arginine | AS | XM_032719 | 922-1101 |
| methyltransferase-1 (CARM1) | | | |
| Casein kinase 1, epsilon (CSNK1E) | S | NM_001893 | 996-1162 |
| Cathepsin D (CTSD) | S | NM_001909 | 1080-1319 |
| CCR7 | AS | NM_001838 | 1214-1389 |
| CD68 | AS | NM_001251 | 234-410 |
| CD74 | AS | NM_004355 | 18-238 |
| CDC-like kinase 3 (CLK3) | S | NM_001292 | 313-424 |
| Cysteine sulfunic acid decarboxylase- | AS | NM_015989 | 676-860 |
| related protein (CSAD) | | | |
| Colony stimulating factor 3 receptor | AS | NM_000760 | 55-234 |
| (CSF3R) | | | |
| Casein kinase 1, gamma 2 | AS | NM_001319 | 87-349 |
| (CSNK1G2) | | | |
| RNA helicase, DECD variant | AS | NM_005804 | 1229-1447 |
| (DDXL) | | | |
| DNA cytosine-5-methyltransferase 3 | AS | NM_022552 | 329-588 |
| alpha (DNMT3A) | | | |
| Dual specificity phosphatase 1 | AS | NM_00417 | 17-245 |
| (DVSP1) | | | |
| G protein-coupled receptor kinase 6 | AS | NM_002082 | 532-765 |
| (GPRK6) | | | |
| Human ADP/ATP translocase | AS | J03592 | 469-658 |
| Leukocyte receptor cluster member 8 | S | XM_044313 | 1182-1357 |
| (LENG8) | | | |
| Mitogen-activated protein kinase 7 | S | NM_005043 | 698-853 |
| (MAP2K7) | | | |
| Macrophage migration inhibitory | AS | NM_002415 | 49-323 |
| factor (MIF) | | | |
| Misshapen/NIK-related kinase | AS | NM_{‘3}015716 | 981-1110 |
| (MINK) | | | |
| Protein expressed in non-metastatic | AS | NM_005009 | 449-646 |
| cells 4 (NME4) | | | |
| Nonreceptor protein-tyrosine kinase | AS | M63877 | 2717-2880 |
| (fgr) | | | |
| P101-PI3K | AS | NM_014308 | 85-286 |
| P2X1 receptor gene | AS | AF078925 | 16-45 |
| Phosphodiesterase 3B (PDE3B) | S | NM_000922 | 12-170 |
| Protein tyrosine kinase 2 beta | AS | NM_004103 | 313-546 |
| (PTK2B) | | | |
| Protein tyrosine phosphatase HD-PTP | S | AN025194 | 2218-2383 |
| (PTPN23) | | | |
| RAB7 | S | X93499 | 447-584 |
| SLC11A1 | AS | NM_000578 | 1163-1399 |
| PI-3-kinase-related (SMG1) | AS | XM_043965 | 4357-4627 |
| Serine/threonine kinase 10 (STK 10) | AS | NM_005990 | 760-900 |
| TAP1 | AS | NM_000593 | 1898-2095 |
| Thromboxane A2 receptor | AS | L14561 | 2404-2580 |
| (TBXA2R) | | | |
| Tyrosine kinase 2 (TYK2) | S | NM_003331 | 18-261 |
| Ubiquitin-conjugating enzyme E2M | AS | NM_003969 | 66-225 |
| (UBE2M) | | | |
| Uridine phosphorylase (UP) | AS | NM_003364 | 1090-1343 |
| Gamma-aminobutyic acid B receptor | S | NM_021905 | 898-1091 |
| 1 (GABBR1) | | | |

AS = Antisense
S = Sense

What we claim is:

1. A method of identifying a compound capable of inhibiting HIV infection in a cell comprising the step of identifying an inhibitor of a target in said human host cell, wherein said target is URF6, URF 2, Squalene synthetase, RTLV associated endogenous retrovirus, Human 2-oxoglutarate dehydrogenase, TCBA, Calnexin, HAUSP, ARF3, eIF4B, eIF3, Glucosidase II, Glucosidase II, Na⁺-D-glucose cotransport regulator, CD47, CD44, BDP-1 tyrosine phosphatase, PI3K, EF-1, Mitochondrial aspartate amino transferase, Double strand break repair gene, guanine nucleotide releasing protein, BTG-1, Lymphocyte specific protein 1, Protein phosphatase 2A, ERF-1, GTP binding protein, Importin beta subunit, L1CAM, HSPG, Zinc finger factor 1, BMP1-6, U-snRNP associated cyclophilin, Recepin, Lipocortin II/Annexin II, hnRNP A1, ArgBP2a, Keratin related protein, Glucosyltransferase, Rox, p18 protein, E1c, Ferritin heavy subunit, p40, MIP-1α, HSP90, MIP-1β, NF-kB binding subunit, BBC-1, α-enolase, TCTP, DAP 5, FK-506 binding protein 1A, TRAP-beta, TID1, HIP, PABP, Cytokine effector-inflammatory response, Nuclear U4A RNA, HnRNP A2/B1, IL-1 beta, TNF-α receptor, HYPK mRNA, HIV-1 TAR binding protein, TRAP-delta, ATP6E, MO25, CD69, Mitochondrial cytochrome oxidase I, Csa-19, 14-3-3 zeta protein, Nip 7-1, EF-1 delta, E16 mRNA, Arginyl tRNA synthetase, Novel nuclear targeted gene, eIF4AII, WBSCRI, C21orf4, Protein phosphatase 2A B56 gamma 1, DAP12, PDCD4, Glutaredoxin, eIF4AI, GA17, MAD-3/ NFKBIA, RANTES, IL-6, FYN binding protein, ABC transporter, HSHIP, IEX-IL, CDC42, Tryptophanyl tRNA synthetase, TRAP-gamma, CXCR-4, Cyclin T1, PDIR, G3PDH, CCR4, GNB2L1, Cathepsin B, Cathepsin L, Vacuolar H+ ATPase proton channel subunit 6C, Prolyl 4-hydroxylase, Protein phosphatase 2A α catalytic, ATP1A1, O-linked GlcNAc transferase, CDP-diacylglycerol synthase 2, FoF1 ATP synthase f subunit, Guanylate binding protein, ATP5G2, Phosphorylase kinase, alpha 2, SOD-2, NADH ubiquinine oxidoreductase B22 subunit, DEAD/H Box 5, DEAD/H 9, Aryl Sulfotransferase, Cytochrome b gene, ATIC, Cytochrome bc-1 core protein, CD11c, HELO1, NPM-RAR, Protein phosphatase I regulatory, Aldehyde dehydrogenase, Glucosamine-6-, phosphate deaminase, DDX3, ATP5E, CAPNS1, CARM1, CSNK1E, CTSD, CCR7, CD68, CD74, CLK3, CSAD, CSF3R, CSNK1G2, DDXL, DNMT3A, DUSP1, GPRK6, Human ADP/ATP translocase, LENG8, MAP2K7, MIF, MINK, NME4, Nonreceptor protein-tyrosine kinase (fgr), P101-PI3K, P2X1 receptor gene, PDE3B, PTK2B, PTPN23, RAB7, SLC11A1, SMG1, STK10, TAP1, TBXA2R, TYK2, UBE2M, UP, or GABBR1.

2. The method, as claimed in claim 1, wherein said target is a validated target involved in HIV infection.

3. The method, as claimed in claim 2, wherein said target is a validated target that is involved in HIV infection, wherein the target has been validated by a process comprising the steps of:
(a) inhibiting said target in a cell by a method selected from the group consisting of gene knock-out, anti-sense oligonucleotide expression target overexpression, viral stage assays, GSE expression and Target protein inhibition assays, and
(b) assaying said cell for the ability of HIV to infect said cell.

4. The method, as claimed in claim 1, wherein said cell is selected from HeLa cells and primary T cells.

5. The method, as claimed in claim 1, wherein said step of identifying a compound comprises the steps of:
(a) contacting a cell with a putative inhibitor; and
(b) assessing inhibition of said target by a method selected from the group consisting of:
(i) assaying for reduced expression of said target; and
(ii) assaying for reduced activity of said target.

6. The method, as claimed in claim 5, wherein expression of said target is measured by polymerase chain reaction.

7. The method, as claimed in claim 5, wherein expression of said target is measured using an antibody immunologically specific for said target.

8. The method, as claimed in claim 5, wherein the activity of said target is measured by measuring the amount of a product generated in a biochemical reaction mediated by said target.

9. The method, as claimed in claim 5, wherein the activity of said target is measured by measuring the amount of a substrate consumed in a biochemical reaction mediated by said target.

10. The method, as claimed in claim 1, wherein said inhibitor is identified by:
(a) determining the three-dimensional structure of said target; and
(b) determining the three-dimensional structure of an inhibitor using computer software capable of modeling the interaction of said target and putative test compounds.

11. The method, as claimed in claim 1, wherein said inhibitor of a target inhibits HIV infection.

12. The method, as claimed in claim 1, wherein said target is a validated target that is involved in HIV infection, wherein the target has been validated by a process comprising the steps of:
(a) inhibiting said target in a human host cell, and
(b) assaying the human host cell for the ability to be infected by HIV.

13. The method, as claimed in claim 12, wherein said human host cell is selected from the group consisting of T cells, macrophages and HeLa cells.

14. An inhibitor of a target conferring resistance to HIV infection.

15. The inhibitor of claim 14, wherein the inhibitor is identified by a method comprising:
(a) contacting said human host cell with a putative inhibitor; and
(b) assessing inhibition of said target by a method selected from the group consisting of:
(i) assaying for reduced expression of said target; and
(ii) assaying for reduced activity of said target.

16. The inhibitor of claim 14, wherein the inhibitor is not toxic to a human host cell that is not infected with HIV.

17. The inhibitor of claim 14, wherein the inhibitor promotes apoptosis in a human host cell infected with HIV.

18. A pharmaceutical composition comprising a therapeutically-effective amount of the inhibitor of claim 14 and a pharmaceutically-acceptable carrier.

19. A method of conferring resistance to HIV infection in an individual, comprising administering to the individual the pharmaceutical composition of claim 18.

20. A method according to claim 12, wherein the target gene in the human host cell is homozygously inactivated.

21. A method according to claim 20, wherein the human host cell is a recombinant cell having the target gene homozygously inactivated by gene knockout.
